(19) United States
(12) Patent Application Publication
(10) Pub. No.: US 2025/0352488 A1
(43) Pub. Date: Nov. 20, 2025

(54) IONIZABLE LIPIDS AND NANOPARTICLES COMPRISING SAME

(71) Applicant: MANA BIO LTD., Tel Aviv (IL)

(72) Inventors: Omer ADIR, Haifa (IL); Galoz KANETI, Haifa (IL); Igor NUDELMAN, Haifa (IL); Aviv ROTMAN, Tel Aviv (IL); Guy D. ROSIN, Tel Aviv (IL); Roy NEVO, Tel Aviv (IL); Yogev DEBBI, Beit Yehoshua (IL); Maya KADURI, Haifa (IL); Lee GOLDFRYD, Nesher (IL)

(21) Appl. No.: 18/872,928

(22) PCT Filed: Jun. 8, 2023
(86) PCT No.: PCT/IL2023/050596

§ 371 Date: Dec. 9, 2024

Related U.S. Application Data
(60) Provisional application No. 63/350,540, filed on Jun. 9, 2022.
(60) Provisional application No. 63/437,800, filed on Jan. 9, 2023.

(51) Int. Cl. A61K 9/51; C07C 217/28

(52) U.S. Cl. CPC A61K 9/5123; C07C 217/28

(57) ABSTRACT

One or more ionizable lipid(s) and lipid nanoparticles comprising same are provided. Pharmaceutical compositions comprising the lipid nanoparticles encapsulating an active agent are also provided.

### CROSS REFERENCE TO RELATED APPLICATIONS

This application claims the benefit of priority under 35 USC § 119(e) of U.S. Provisional Patent Application Nos. 63/350,540, filed Jun. 9, 2022, entitled “IONIZABLE LIPIDS AND NANOPARTICLES COMPRISING SAME”, and 63/437,800, filed Jan. 9, 2023, entitled “IONIZABLE LIPIDS AND NANOPARTICLES COMPRISING SAME” the contents of which are incorporated herein by reference in their entirety.

### FIELD OF INVENTION

The present invention is directed to ionizable lipids and lipid nanoparticles comprising same and use thereof in pharmaceutical compositions.

### BACKGROUND OF THE INVENTION

New delivery methods for therapeutic and diagnostic compounds are in constant development. Although lipid-based nanoparticles are a well-known delivery modality, these agents are also constantly undergoing improvement. Among other concerns, the ability of a therapeutic carrier to effectively load and subsequently deliver the active agent to a target site, is of great importance for reduced dosing, and improved treatment efficiency.

Although various ionizable lipids capable of encapsulation of hydrophilic agents such as DNA and/or RNA are known, there is a constant need for new and superior ionizable lipids. In particular there is a great need for development of new and superior ionizable lipids, which are capable of enhancing drug delivery to specific locations in the body.

### SUMMARY OF THE INVENTION

The present invention provides new compounds suitable for use as ionizable lipids. In addition, nanoparticles comprising same are provided. Compositions comprising the nanoparticles, which are useful for delivery of an active agent to a subject such as for treating or preventing a disease or disorder within the subject are also provided.

According to a first aspect, there is provided a compound represented by Formula I:

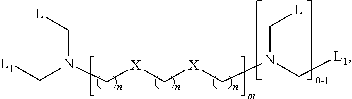

wherein: each L is independently R1,

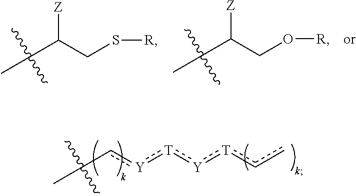

each L1 is independently R1,

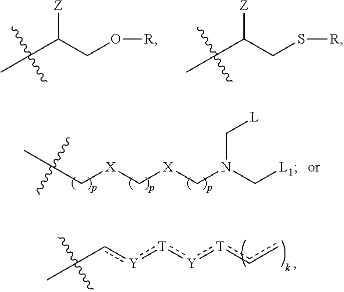

[symbol]

represents a single bond, a triple bond or a double bond; Z represents independently —OH or —SH; each k is independently between 0 and 10 or between 1 and 24, including any range between; each Y independently is absent or comprises CH₂, CHR′₂, NR′₂, NH, O, S, —CONH—, —CONR′—, —C(═NH)NR′—, —C(═S)NR′—, —NC(═O)—, —NC(═O)O—, —NC(═O)N—, —NC(═S)O—, —NC(═S)N—, —C(═O)—, —C(═O)O—, —OC(═O)O—, —OC(═O)N—, —OC(═S)O—, —OC(═S)N—, or phosphate, as allowed by valency; each T independently represents an optionally substituted C5-C30 alkyl or an optionally substituted C5-C30 alkenyl; each R′ is independently H or comprises an optionally substituted C₁-C₁₀ alkyl, an C₁-C₁₀ alkyl-aryl, an C₁-C₁₀ alkyl-cycloalkyl, optionally substituted C₃-C₁₀ cycloalkyl, optionally substituted C₃-C₁₀ heterocyclyl, optionally substituted heteroaryl, optionally substituted aryl or a combination thereof; each X independently represents a heteroatom, CH₂, an optionally substituted C1-C10 alkyl, or X is absent; each n and p is independently between 0 and 5, and at least one n is not 0; m is between 1 and 3; each R independently is H, or comprises an optionally substituted C5-C30 alkyl; each R1 is an optionally substituted C1-C24 alkyl, and at least one L or L1 is or comprises

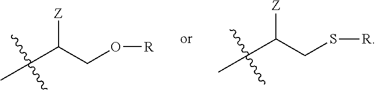

In one embodiment, any one of R and R1 further comprises at least one unsaturated bond.

In one embodiment, the heteroatom comprises O, N, NH, NR1, S, or a phosphate group.

In one embodiment, each L is independently

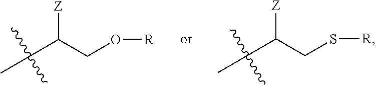

In one embodiment, each X independently is O, or is absent.

In one embodiment, any one of R and R1 represents a linear or a branched C1-C24 or C1-C10 alkyl.

In one embodiment, the compound is represented by Formula II:

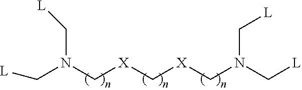

wherein each L is independently R1,

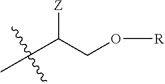

or; and wherein at least one L is

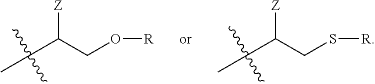

In one embodiment, the compound is characterized by pKa value between 5 and 9.

In one embodiment, the compound comprises any one of the compounds of Example 1, or Example 4.

In another aspect, there is provided a lipid nanoparticle comprising the compound of the invention, and an active agent.

In one embodiment, an average size of the lipid nanoparticle is in a range between 50 and 300 nm.

In one embodiment, the active agent comprises a polynucleic acid.

In one embodiment, the lipid nanoparticle further comprises a lipid, wherein the lipid comprises a helper lipid, and optionally comprises a structural lipid, a modified lipid, or any combination thereof.

In one embodiment, a weight ratio between (i) the total amount of the compound and of the lipid, and (ii) the polynucleic acid within the lipid nanoparticle is between 0.001:1 and 10:1.

In one embodiment, the lipid comprises the helper lipid, the modified lipid, and a sterol.

In one embodiment, a ratio of the compound relative to the total lipid content of the lipid nanoparticle is between 10 and 80 mol %.

In another aspect, there is provided a pharmaceutical composition comprises a plurality of the lipid nanoparticles of the invention and a pharmaceutically acceptable carrier.

In one embodiment, the pharmaceutical composition comprising an effective amount of the active agent.

In one embodiment, the pharmaceutical composition is formulated for systemic administration to a subject, local administration to a subject, or both.

In one embodiment, the pharmaceutical composition is for use in the treatment of a disease or disorder in a subject in need thereof.

In another aspect, there is provided a method for delivering an active agent to a tissue of a subject, the method comprising administering to the subject an effective amount of the pharmaceutical composition of the invention, thereby delivering the active agent to the tissue.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIG. 1. presenting a bar graph expression analysis in-vivo of three LNP composition a. FMB-1050. FMB-428 and FMB-389, within a liver tissue compared to a heart tissue, a spleen tissue, a kidney tissue, and a lung tissue.

FIG. 2. presenting a bar graph expression analysis in-vivo of three LNP composition a. FMB-1143, FMB-748 and 745, within a lung tissue compared to a heart tissue, a spleen tissue, a kidney tissue, and a liver tissue.

### DETAILED DESCRIPTION OF THE INVENTION

Compounds disclosed by the present invention, were discovered by methods of computational screening. Ionizable lipid candidates were generated in-silico and ranked based on their predicted activity using a machine learning algorithm. After several optimization cycles in silico, a chemical library containing several molecules was obtained. The disclosed compounds were selected based on results obtained from in-vitro experiments, as represented hereinbelow (Examples section).

Compounds with at least 30% encapsulation efficiency (such as at least 80% encapsulation efficiency) and which induced at least about 10 times greater intracellular expression and/or cellular internalization of an RNA sequence, compared to transfection with Lipofectamine 2000 (see Example section), were selected as suitable ionizable lipid candidates for further in-vivo studies.

According to a first aspect, there is provided a compound comprising an ionizable moiety (e.g., head group) covalently bound to a lipophilic tail (e.g., hydrocarbon-based chain), wherein the ionizable moiety is represented by any one of Formulae:

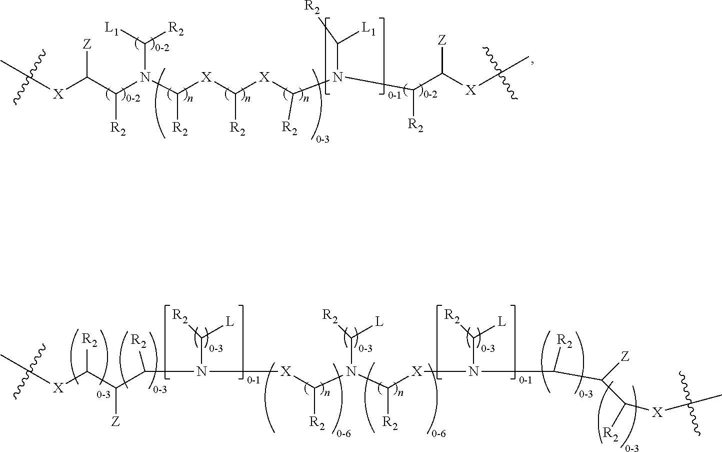

wherein a wavy bond represents an attachment point to H, L1 or to the lipophilic tail; wherein each L₁, X and n independently is as described hereinbelow, and wherein each R2 independently represents H, or one or more substituents as described herein. In some embodiments, each L and/or X represents the same or different chemical moiety. In some embodiments, each n represents the same or different numerical value or range. In some embodiments, at least one wavy bond represents an attachment point to the lipophilic tail.

In some embodiments, the lipophilic tail comprises between 10 and 50 carbon atoms (either straight, branched or cyclic hydrocarbon chain), and optionally comprises one or more unsaturated bonds. In some embodiments, the compound is an amphiphilic compound. In some embodiments, the compound is capable of spontaneously self-assembling to form a nanoparticle (e.g., a lipid nanoparticle) in an aqueous solution.

In some embodiments, the ionizable moiety is capable of undergoing ionization (protonation, or positive ionization) within a solution having a pH value below the pKa value of the ionizable moiety. In some embodiments, the ionizable moiety is capable of undergoing protonation within a solution having a pH value below the pKa value of the ionizable moiety. In some embodiments, at least 50 mol % of the ionizable moieties are positively charged (or protonated) within a solution having a pH value below the pKa value of the ionizable moiety.

In some embodiments, the pKa value of the ionizable moiety is between 5 and 9, including any range between. In some embodiments, the pKa value of the ionizable moiety is between 5 and 8, between 6 and 8, between 6 and 7, between 7 and 9, between 6 and 9, including any range between.

In some embodiments, the ionizable moiety is bound to the lipophilic tail via a spacer or via a covalent bond.

In some embodiments, the lipophilic tail comprises one or more moieties represented by Formula:

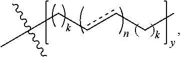

### or by Formula

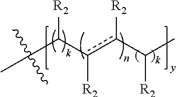

wherein: [symbol]
represents a single bond, a double or a triple bond; R2 is as described herein; k and n, are integers each independently being between 1 and 10, or between 1 and 24, including any range between; and y is between 1 and 3.

In some embodiments, the compound of the invention has a MW of between 100 and 2000 Da, between 100 and 300 Da, between 100 and 500 Da, between 100 and 800 Da, between 300 and 500 Da, between 100 and 1,000 Da, between 500 and 800 Da, between 500 and 2000 Da, between 500 and 1,000 Da, between 800 and 1,000 Da, between 800 and 1,500, between 1,000 and 2,000, including any range between.

In another aspect, there is provided a compound and/or a salt thereof, wherein the compound is represented by Formula 1:

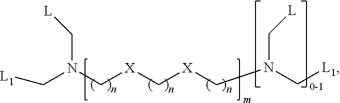

### or by Formula 1A:

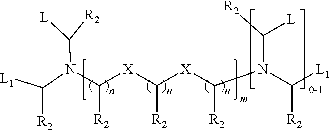

wherein: each L is independently R1,

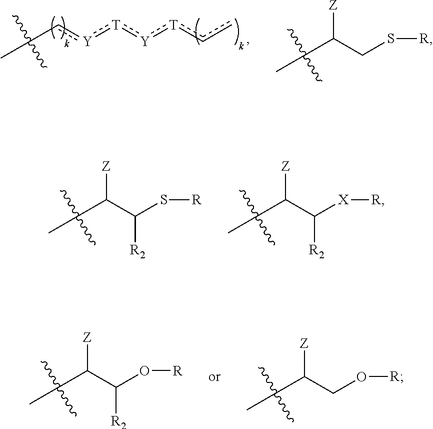

each L1 is independently R1,

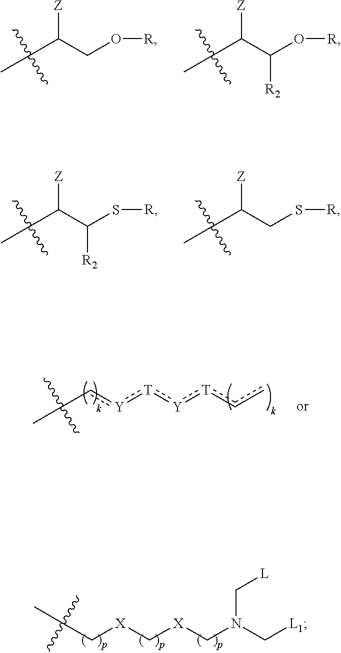

each X independently represents a heteroatom, CH2, an optionally substituted C1-C10 alkyl, or X is absent (i.e., is a bond); each n and p is independently between 0 and 5, and at least one n is not 0; m is between 1 and 3; Z represents independently —OH or —SH; each R independently is H, or comprises an optionally substituted C5-C30 alkyl, or an optionally substituted C1-C30 alkyl; and each R1 is an optionally substituted C1-C24 alkyl, an optionally substituted C1-C24 alkylhydroxy, (optionally substituted C1-C10alkyl)-X(R′)— and at least one of L and L1 is or comprises

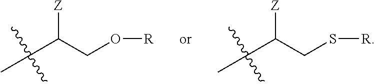

In some embodiments, any one of R and R1 further comprises at least one unsaturated bond.

In some embodiments, the compound of the invention is represented by Formula:

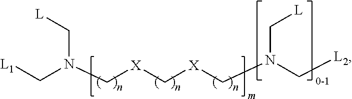

wherein each L1 and L2 is independently R1,

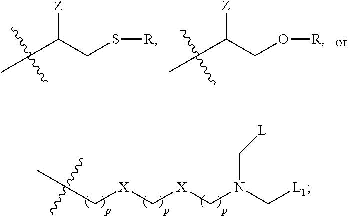

wherein X, Z, L, R, R1, p, n, m are as described herein; and wherein at least one of L and L1 is or comprises

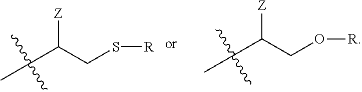

In some embodiments, each n and p is independently between 0 and 5, between 0 and 3, between 0 and 2, (e.g., 0, 1, 2, 3, 4 or 5) and at least one n is not 0 (e.g., 1, 2, 3, 4 or 5); m is between 1 and 3 (e.g., 1, 2, or 3), including any combination thereof.

In some embodiments, R is represented by Formula:

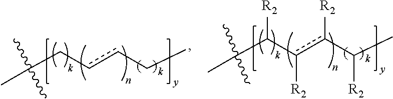

### or by Formula:

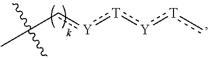

wherein: [symbol] represents a single bond, a triple bond or a double bond; k and n, are integers each independently being between 1 and 10, or between 1 and 24, including any range between; Z represents independently —OH or —SH; and y is between 1 and 3; and each Y is absent or independently comprises CH₂, CHR′₂, NR′₂, NH, O, S, —CONH—, —CONR′—, —C(═NH)NR′—, —C(═S)NR′—, —NC(═O)—, —NC(═O)O—, —NC(═O)N—, —NC(═S)O—, —NC(═S)N—, —C(═O)—, —C(═O)O—, —OC(═O)O—, —OC(═O)N—, —OC(═S)O—, —OC(═S)N—, or phosphate, as allowed by valency; and wherein each T independently represents an optionally substituted C5-C30 alkyl or an optionally substituted C5-C30 alkenyl. In some embodiments each R′ is independently H or comprises an optionally substituted C₁-C₁₀ alkyl, an C₁-C₁₀ alkyl-aryl, an C₁-C₁₀ alkyl-cycloalkyl, optionally substituted C₃-C₁₀ cycloalkyl, optionally substituted C₃-C₁₀ heterocyclyl, optionally substituted heteroaryl, optionally substituted aryl or a combination thereof, or R′ is absent, as allowed by valency.

In some embodiments, the heteroatom comprises O, N, NH, NR₁, or S. In some embodiments, each X independently is O, or is absent.

In some embodiments, one of R and R1 each independently represents a linear or a branched alkyl.

In some embodiments, L is

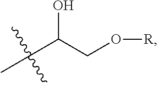

wherein R is as described herein. In some embodiments, each R represents the same or different alkyl.

In some embodiments, L is

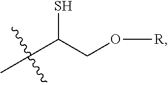

wherein R is as described herein. In some embodiments, each R represents the same or different alkyl.

In some embodiments, L is

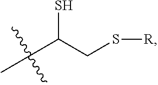

wherein R is as described herein. In some embodiments, each R represents the same or different alkyl.

In some embodiments, L is

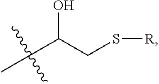

wherein R is as described herein. In some embodiments, each R represents the same or different alkyl.

As used herein, the term “alkyl” describes an aliphatic hydrocarbon including straight chain and branched chain groups. In some embodiments, the alkyl group has 1 to 10 carbon atoms, 1 to 30 carbon atoms, 1 to 24, or 5-30 carbon atoms. In some embodiments, the alkyl group is a C1-C6 alkyl. In some embodiments, the alkyl group is a C1-C6 alkyl, C1-C10 alkyl, C1-C8 alkyl, C5-C30 alkyl, C5-C24 alkyl, C5-C20 alkyl, C5-C10 alkyl, C8-C30 alkyl, C8-C24 alkyl, C8-C15 alkyl, C8-C20 alkyl, C8-C12 alkyl, including any range between. Whenever a numerical range e.g., “5-30”, is stated herein, it implies that the group, in this case the alkyl group, may contain 5 carbon atoms, 6 carbon atoms, 10 carbon atoms, between 5 and 20, between 5 and 25, between 5 and 30, between 10 and 20, between 10 and 25, between 10 and 30, including any range between, up to and including 30 carbon atoms. The alkyl can be substituted or unsubstituted, as defined herein.

The term “alkyl”, as used herein, also encompasses saturated or unsaturated hydrocarbon, hence this term further encompasses alkenyl and alkynyl.

The term “alkenyl” describes an unsaturated alkyl, as defined herein, having between 2 and 30 carbon atoms and at least one carbon-carbon double bond. The alkenyl may be substituted or unsubstituted by one or more substituents, as described hereinabove.

The term “alkynyl”, as defined herein, is an unsaturated alkyl having between 2 and 30 carbon atoms and at least one carbon-carbon triple bond. The alkynyl may be substituted or unsubstituted by one or more substituents, as described hereinabove.

In some embodiments, the alkyl group is a C1-C10 alkyl or a C1-C6 alkyl.

As used herein the term “C1-C10 alkyl” including any C1-C10 alkyl related compounds, is referred to any linear or branched alkyl chain comprising between 1 and 6, between 1 and 2, between 2 and 3, between 3 and 4, between 4 and 5, between 5 and 6, between 6 and 7, between 7 and 8, between 8 and 9, between 9 and 10 carbon atoms, including any range therebetween. In some embodiments, C1-C10 alkyl comprises any of methyl, ethyl, propyl, butyl, pentyl, iso-pentyl, hexyl, heptyl, octyl, nonyl, decyl and tert-butyl or any combination thereof. In some embodiments, C1-C10 alkyl as described herein further comprises an unsaturated bond, wherein the unsaturated bond is located at 1^{st}, 2^{nd}, 3^{rd}, 4^{th}, 5^{th}, 6^{th}, 7^{th}, 8^{th}, 9^{th}, or 10^{th} position of the C1-C10 alkyl.

As used herein the term “C1-C6 alkyl” including any C1-C6 alkyl related compounds, is referred to any linear or branched alkyl chain comprising between 1 and 6, between 1 and 2, between 2 and 3, between 3 and 4, between 4 and 5, between 5 and 6, carbon atoms, including any range therebetween. In some embodiments, C1-C6 alkyl comprises any of methyl, ethyl, propyl, butyl, pentyl, iso-pentyl, hexyl, and tert-butyl or any combination thereof. In some embodiments, C1-C6 alkyl as described herein further comprises an unsaturated bond, wherein the unsaturated bond is located at 1st, 2nd, 3rd, 4th, 5th, or 6th position of the C1-C6 alkyl.

In some embodiments, the compound of the invention is represented by Formula II:

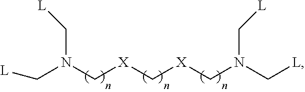

wherein each of L, X, Z and n is independently as described hereinabove, and wherein at least one L is or comprises

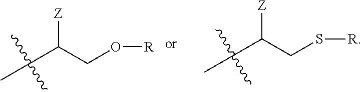

In some embodiments, the compound of the invention is represented by Formula III:

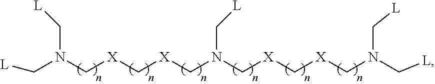

wherein each of L, X and n independently is as described hereinabove and wherein at least one L is or comprises

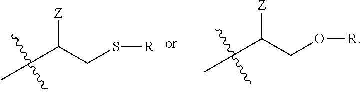

In some embodiments, the compound of the invention is represented by any one of Formulae disclosed herein, and wherein each L is the same or different.

In some embodiments, the compound of the invention is represented by Formulae I to III, wherein the sum of all n within the molecule is between 2 and 15, between 2 and 10, between 2 and 8, including any range between.

In some embodiments, the compound of the invention is represented by Formula IIA:

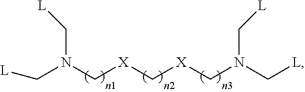

wherein each X is independently O or is absent; each of n1, n2, and n3 is independently between 0 and 3 and wherein a sum of n1, n2, and n3 is between 2 and 15, between 2 and 10, between 2 and 8, including any range between, and L is as described herein; and wherein at least one L is or comprises

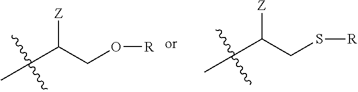

wherein R is as described hereinabove, and wherein Z is OH. In some embodiments, the compound of the invention is represented by Formula IIA, wherein each L is

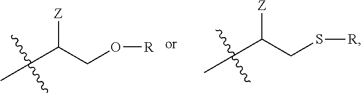

and R is as described hereinabove, and wherein Z is OH. In some embodiments, the compound of the invention is represented by Formula IIA, wherein each X is absent and a sum of n1, n2, and n3 is between 2 and 15, between 2 and 10, between 2 and 8, including any range between. In some embodiments, the compound of the invention is represented by Formula IIA, wherein each X represents O, n1 is between 1 and 3; and each of n2, and n3 is independently between 1 and 5, between 1 and 3, and wherein a sum of n1, n2, and n3 is between 2 and 15, between 2 and 10, between 2 and 8, including any range between.

In some embodiments, the compound of the invention is represented by Formula IIIA:

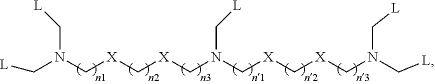

wherein each X is independently O or is absent; each of n1, n′1, n2, n′2, n3 and n′3 is independently between 0 and 3, wherein a sum of n1, n2, and n3 is between 2 and 15, between 2 and 10, between 2 and 8, including any range between; and wherein a sum of n′1, n′2, and n′3 is between 2 and 15, between 2 and 10, between 2 and 8, including any range between, and L is as described herein, and wherein at least one L is or comprises

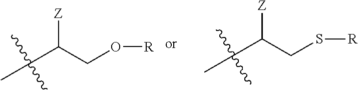

wherein R is as described hereinabove, and wherein Z is OH. In some embodiments, the compound of the invention is represented by Formula IIIA, wherein each L is

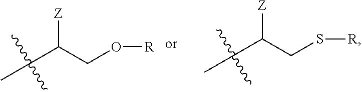

and wherein R is as described hereinabove, and wherein Z is OH.

In some embodiments, the compound of the invention is represented by Formula IIIB:

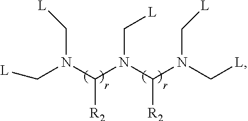

wherein each r independently represents an integer between 2 and 15, between 2 and 10, between 2 and 8, including any range between, and each L and R2 is independently as described herein.

In some embodiments, the compound of the invention is represented by Formula IIIA, wherein each X is O; each n1 and n′1 is independently between 0 and 3, n2, n′2, n3 and n′3 is independently between 0 and 3, wherein a sum of n1, n2, and n3 is between 2 and 15, between 2 and 10, between 2 and 8, including any range between; and wherein a sum of n′1, n′2, and n′3 is between 2 and 15, between 2 and 10, between 2 and 8, including any range between; and wherein each L is

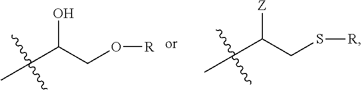

and R is as described hereinabove.

In some embodiments, the compound of the invention is represented by any one of the Formulae disclosed herein, wherein at least one L is

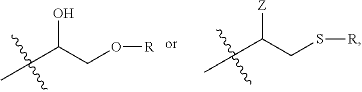

or wherein each L is L is

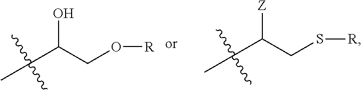

and R is as described hereinabove.

In some embodiments, the compound of the invention comprises any one of the compounds of Example 1, including any salt, any tautomer, and/or any stereoisomer (e.g., an enantiomer, and/or a diastereomer) thereof.

As used herein, the term “substituted” or the term “substituent” are related to one or more (e.g., 2, 3, 4, 5, or 6) substituents, wherein the substituent(s) is as described herein.

As used herein, the term substituent comprises halogen, —NO₂, —CN, —OH, —CONH₂, —CONR′₂, —CNNR′₂, —CSNR′₂, —CONH—OH, —CONH—NH₂, —NHCOR, —NHCSR, —NHCNR, —NC(═O)OR, —NC(═O)NR′, —NC(═S) OR′, —NC(═S)NR′, —SO₂R′, —SOR′, —SR′, —SO₂OR′, —SO₂N(R′)₂, —NHNR′₂, —NNR′, C₁-C₆ haloalkyl, optionally substituted C₁-C₆ alkyl, —NH₂, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′₂, C₁-C₆ alkyl-SR′, —CONH(C₁-C₆ alkyl), —CON(C₁-C₆ alkyl)₂, —CO₂H, —CO₂R′, —OCOR, —OCOR′, —OC(═O) OR′, —OC(═O)NR′, —OC(═S) OR′, —OC(═S)NR′, or a combination thereof; wherein each R′ independently represents hydrogen, or is selected from the group comprising optionally substituted C₁-C₁₀ alkyl, optionally substituted C₃-C₁₀ cycloalkyl, optionally substituted C₃-C₁₀ heterocyclyl, optionally substituted heteroaryl, optionally substituted aryl, or a combination thereof.

As used herein, the term “C₁-C₆ haloalkyl” refers to C₁-C₆ alkyl as described herein substituted by 1, 2, 3, 4, 5, 6, 7, 8, 9, 10 halide atoms, wherein halide is selected from F, Br, Cl, and I, or a combination thereof.

As used herein the term “(C₃-C₁₀) cycloalkyl” is referred to an optionally substituted C3, C4, C5, C6, C7, C8, C9 or C10 ring. In some embodiments, (C₃-C₁₀) ring comprises optionally substituted cyclopropane, cyclobutene, cyclopentane, cyclohexane, or cycloheptane.

As used herein the term “C₃-C₁₀ heterocyclyl” is referred to an optionally substituted C3, C4, C5, C6, C7, C8, C9 or C10 heterocyclic aromatic and/or aliphatic, or unsaturated ring.

In some embodiments, the term “hydroxy(C₁-C₆ alkyl)” and the term “C₁-C₆ alkoxy” are used herein interchangeably and refer to C₁-C₆ alkyl as described herein substituted by 1, 2, 3, 4, 5, 6, 7, 8, 9, 10 hydroxy group(s), wherein the hydroxy group(s) is located at 1^{st}, 2^{nd}, 3^{rd}, 4^{th}, 5^{th}, or 6^{th} position of the C₁-C₆ alkyl, including any combination thereof.

In some embodiments, the compound of the invention substantially comprises a single enantiomer of any one of the compounds described herein, wherein substantially is at least 70%, at least 75%, at least 80%, at least 85%, at least 90%, at least 92%, at least 93%, at least 95%, at least 97%, at least 98%, at least 99% by weight, including any value therebetween.

In some embodiments, the compound of the invention further encompasses any structurally similar functional derivative of the compounds disclosed herein, wherein structurally similar is at least 90%, at least 95%, at least 96%, at least 97%, at least 98%, at least 99% structure similarity, including any range between.

In some embodiments, a functional derivative refers to an ionizable lipid having a pKa value between 6.2 and 6.8, and capable of undergoing self-assembly in water so as to stably bind and/or encapsulate a polynucleic acid. In some embodiments, a functional derivative is further configured of cell internalizing a polynucleic acid (e.g., by forming a lipid nanoparticle as described herein). Cellular internalization can be determined as described hereinbelow.

In some embodiments, the term “structure similarity” refers to a fingerprint similarity between two molecules. The term “fingerprint similarity” is well-understood by a skilled artisan. In some embodiments, the fingerprint similarity is calculated based on circular fingerprints, substructure keys-based fingerprints, and/or topological or path-based fingerprints.

Exemplary circular fingerprints include but are not limited to: Molprint 2D, ECFP (or Morgan fingerprint), FCFP, etc. In some embodiments, the term “structure similarity” as used herein, is calculated by Morgan fingerprint.

### Carriers

In another aspect, there is provided a carrier for an active agent(s), wherein the carrier is in a form of a nanoparticle. In some embodiments, the carrier encapsulates the active agent within the core. In some embodiments, the active agent is a small molecule and/or a biologic molecule, such as polypeptide, a polynucleotide, etc. In some embodiments, the active agent is water soluble (e.g. having water solubility of at least 0.1 g/L at a temperature between 2° and 30° C.). In some embodiments, the active agent is selected from a therapeutic agent, a prophylactic agent and a diagnostic agent including any combination thereof. In some embodiments, the one or more active agents are selected from the group consisting of: a protein, a peptide, a nucleic acid, a small molecule, and an antibody.

In some embodiments, the carrier is in the form of a lipid nanoparticle comprising the compound of the invention, and the active agent. In some embodiments, the lipid nanoparticle comprises a shell and an aqueous core, comprising the active agent. In some embodiments, the shell of the lipid nanoparticle comprises the compound of the invention. In some embodiments, the shell of the lipid nanoparticle further comprises a lipid, a sterol, and/or a PEG-lipid, or any combination thereof. In alternative embodiments, the carrier is in the form of a lipid nanoparticle comprising the compound of the invention, a lipid, and the active agent. In some embodiments, lipid nanoparticle comprises is in a form of a core-shell nanoparticle, wherein the shell of the nanoparticle comprises a lipid, and at least one compound of the invention. In some embodiments, the compound of the invention is bound (e.g., via electrostatic interactions) to the active agent (e.g., a polynucleotide).

In some embodiments, under suitable conditions at least one compound of the invention, and optionally the lipid (and optionally the active agent) spontaneously undergo self-assembly in an aqueous solution, so as to form the lipid nanoparticle. In some embodiments, the term “lipid nanoparticle” refers to a nanoparticle (e.g., substantially spherical particle), wherein the shell of the nanoparticle comprises one or more compounds of the invention and optionally one or more lipids (e.g., a helper lipid, such as a cationic lipid, non-cationic lipid; and optionally a sterol, and/or a PEG-modified lipid). Preferably, the lipid nanoparticles are formulated to deliver one or more agents to one or more target cells.

In some embodiments, the nanoparticle has a spherical geometry or shape. In some embodiments, the nanoparticle has an inflated or a deflated shape. In some embodiments, a plurality of core-shell particles is devoid of any characteristic geometry or shape. In some embodiments, the nanoparticle has a spherical shape, a quasi-spherical shape, a quasi-elliptical sphere, a deflated shape, a concave shape, an irregular shape, or any combination thereof.

In some embodiments, the plurality of core-shell particles is substantially spherically shaped, wherein substantially is as described herein. In some embodiments, the plurality of core-shell particles is substantially elliptically shaped, wherein substantially is as described herein. One skilled in the art will appreciate that the exact shape of each of the plurality of core-shell particles may differ from one particle to another. Moreover, the exact shape of the nanoparticle may be derived from any of the geometric forms listed above, so that the shape of the particle does not perfectly fit a specific geometrical form. One skilled in the art will appreciate that the exact shape of the nanoparticle may have substantial deviations (such as at least 5%, at least 10%, at least 20% deviation) from a specific geometrical shape (e.g., a sphere or an ellipse).

In some embodiments, the lipid comprises a helper lipid. In some embodiments, the lipid comprises, a structural lipid, a PEG-lipid or both. In some embodiments, the lipid comprises a helper lipid, and optionally comprises a structural lipid, and/or, a PEG-lipid. In some embodiments, the term “structural lipid” encompasses a non-liposome forming lipid, as described herein. In some embodiments, the structural lipid is or comprises a sterol.

In some embodiments, the helper lipid is or comprises a phospholipid. In some embodiments, the helper lipid is or comprises a liposome forming lipid. As used herein, the term “liposome forming lipid” encompasses lipids (e.g., phospholipids) which upon dispersion or dissolution thereof in an aqueous solution at a temperature above a transition temperature (Tₘ), undergo self-assembly so as to form stable vesicles (e.g., lipid nanoparticles). The terms “liposome forming lipid” and “lipid nanoparticle forming lipid” are used herein interchangeably. As used herein, the term Tm refers to a temperature at which the lipids undergo phase transition from solid (ordered phase, also termed as a gel phase) to a fluid (disordered phase, also termed as fluid crystalline phase). Tm also refers to a temperature (or to a temperature range) at which the maximal change in heat capacity occurs during the phase transition.

In some embodiments, the phospholipid encompasses a single phospholipid specie or a plurality of chemically distinct phospholipids.

In some embodiments, the liposome forming lipid is a phospholipid having one or two C12 to C24 hydrocarbon tails, typically, acyl, alkyl or alkenyl chain) and have varying degrees of unsaturation, from being fully saturated to being fully, partially or non-hydrogenated lipids (the level of saturation may affect rigidity of the liposome thus formed (typically liposomes formed from lipids with saturated chains are more rigid than liposomes formed from lipids of same chain length in which there are un-saturated chains, especially having cis double bonds). In some embodiments, at least one of the liposome forming lipid is a phospholipid having one or two C12 to C20, C16 to C20, or C16 to C18 hydrocarbon tails, including any value and range therebetween. In some embodiments, the liposome forming lipid is fully saturated, linear, or branched.

Further, the phospholipid may be of natural source (e.g., naturally occurring phospholipids), semi-synthetic or fully synthetic lipid, as well as electrically neutral (e.g., zwitterionic), negatively, or positively charged.

Non-limiting examples of neutral phospholipids include but are not limited to diacylphosphatidylcholines, dialkylphosphatidylcholines, sphingomyelins, and diacylphosphatidylethanolamines. Phosphatidylcholines (PC), including those obtained from egg, soybeans, or other plant sources or those that are partially or wholly synthetic, or of variable lipid chain length and unsaturation are suitable for use in the present compositions. Synthetic, semisynthetic, and natural product phosphatidylcholines including, but not limited to, POPC, DOPC, DMPC, distearoylphosphatidylcholine (DSPC), hydrogenated soy phosphatidylcholine (HSPC), soy phosphatidylcholine (soy PC), egg phosphatidylcholine (egg PC), hydrogenated egg phosphatidylcholine (HEPC), and dipalmitoylphosphatidylcholine (DPPC) are suitable phosphatidylcholines for use in the preparation of liposomes. Charged phospholipids can include phosphatidylglycerols, cardiolipins, or headgroup modified lipids such as N-succinyl-phosphatidylethanolamines, N-glutaryl-phosphatidylethanolamines, and PEG-derivatized phosphatidylethanolamines.

Non-limiting examples of cationic lipids or ionizable cationic lipids include but are not limited to 5-carboxyspermylglycinedioctadecylamide or “DOGS,” N-[1-(2,3-dioleyloxy)propyl]-N,N,N-trimethylammonium chloride or “DOTMA”, 2,3-dioleyloxy-N-[2(spermine-carboxamido)ethyl]-N,N-dimethyl-1-pr-opanaminium or “DOSPA”, 1,2-Diolcoyl-3-Dimethylammonium-Propane or “DODAP”, 1,2-Dioleoyl-3-Trimethylammonium-Propane or “DOTAP”. Contemplated cationic lipids also include 1,2-distearyloxy-N,N-dimethyl-3-aminopropane or “DSDMA”, 1,2-dioleyloxy-N,N-dimethyl-3-aminopropane or “DODMA”, 1,2-dilinoleyloxy-N,N-dimethyl-3-aminopropane or “DLinDMA”, 1,2-dilinolenyloxy-N,N-dimethyl-3-aminopropane or “DLenDMA”, N-dioleyl-N,N-dimethylammonium chloride or “DODAC”, N,N-distearyl-N,N-dimethylammonium bromide or “DDAB”, N-(1,2-dimyristyloxyprop-3-yl)-N,N-dimethyl-N-hydroxyethyl ammonium bromide or “DMRIE”, 3-dimethylamino-2-(cholest-5-en-3-beta-oxybutan-4-oxy)-1-(cis,cis-9,12-oc-tadecadienoxy)propane or “CLinDMA”, 2-[5′-(cholest-5-en-3-beta-oxy)-3′-oxapentoxy)-3-dimethyl-1-(cis,cis-9′,1-2′-octadecadienoxy)propane or “CpLinDMA”, N,N-dimethyl-3,4-dioleyloxybenzylamine or “DMOBA”, 1,2-N,N′-dioleylcarbamyl-3-dimethylaminopropane or “DOcarbDAP”, 2,3-Dilinoleoyloxy-N,N-dimethylpropylamine or “DLinDAP”, 1,2-N,N′-Dilinoleylcarbamyl-3-dimethylaminopropane or “DLincarbDAP”, 1,2-Dilinoleoylcarbamyl-3-dimethylaminopropane or “DLinCDAP”, 2,2-dilinoleoyl-4-dimethylaminomethyl-[1,3]-dioxolane or “DLin-K-DMA”, 2,2-dilinoleyl-4-dimethylaminoethyl-[1,3]-dioxolane or “DLin-K-XTC2-DMA”, and 2-(2,2-di((9Z,12Z)-octadeca-9,12-dien-1-yl)-1,3-dioxolan-4-yl)-N,N-di-methylethanamine (DLin-KC2-DMA)).

In some embodiments, the helper lipid is or comprises a non-cationic lipid. As used herein, the term “non-cationic lipid” refers to any neutral, or zwitterionic lipid. Non-cationic lipids include, but are not limited to, dioleoylphosphatidylethanolamine (DOPE), distearoylphosphatidylcholine (DSPC), hydrogenated soy phosphatidylcholine (HSPC), dioleoylphosphatidylcholine (DOPC), dipalmitoylphosphatidylcholine (DPPC), diolcoylphosphatidylglycerol (DOPG), dipalmitoylphosphatidylglycerol (DPPG), palmitoylolcoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), or a mixture thereof.

In some embodiments, the helper lipid is or comprises DOPE, DSPC, POPE, or any combination thereof.

In some embodiments, the helper lipid is a cationic lipid. In some embodiments, the cationic lipid is or comprises any of DOTAP, DDAB, 1,2-dioleoyl-sn-glycero-3-ethylphosphocholine, DPPCethyl (EPC 16:0, or 1,2-dipalmitoyl-sn-glycero-3-ethylphosphocholine) or any combination thereof.

In some embodiments, the PEG-lipid comprises a single PEG moiety covalently bound to the head group of the lipid. In some embodiments, the PEG-lipid comprises a plurality of PEG moieties covalently bound to the head group of the lipid. In some embodiments, the PEG moiety comprises an alkylated PEG such as methoxy poly(ethylene glycol) (mPEG). The PEG moiety can have a molecular weight of the head group from about 750 Da to about 20,000 Da, at times, from about 750 Da to about 12,000 Da and typically between about 1,000 Da to about 5,000 Da, including any range between.

In some embodiments, the term “non-liposome forming lipid” is to be understood as referring to a lipid that does not spontaneously form into a vesicle when brought into an aqueous medium.

There are various types of lipids that do not spontaneously vesiculate and yet are used or can be incorporated into vesicles. In some embodiments, the non-liposome forming lipid is or comprises a sterol.

Non-limiting examples of sterols include but are not limited to: β-sitosterol, β-sitostanol, stigmasterol, stigmastanol, campesterol, campestanol, ergosterol, avenasterol, brassicasterol, fucosterol, cholesterol (Chol), cholesteryl hemisuccinate, and cholesteryl sulfate including any salt or any combination thereof.

In some embodiments, the structural lipid comprises any one of Betulin, Brassicasterol, Calcipotriol, campesterol, cholesterol, Daucosterol, DC-cholesterol, Dehydroergosterol, DMAPC-Chol, DMHAPC-Chol, ergosterol, Fucosterol, HAPC-Chol, Lupeol, MHAPC-Chol, OH—C-Chol, OH-Chol, Oleanolic acid, stigmastanol, stigmasterol, Ursolic acid, a hydrophobic vitamin (e.g. Vitamin D2, Vitamin D3, vitamin E, etc.), β-sitosterol, β-Sitosterol-Acetate, β-sitosterol-arginine, β-sitosterol-cysteine, β-sitosterol-glycine, β-sitosterol-histidine, β-sitosterol-serine, or a steroid, including any salt or any combination thereof.

In some embodiments, a molar concentration of one or more compounds of the invention within the nanoparticle is between 10 and 80 mol %, between 15 and 55 mol %, between 10 and 20 mol %, between 20 and 60 mol %, between 10 and 60 mol %, between 20 and 40 mol %, between 40 and 60 mol %, between 60 and 80 mol %, including any range between. As used herein, the term “concentration” or “molar concentration” refers to a molar ratio relative to the total lipid content of the nanoparticle. In some embodiments, the total lipid content refers to the combined content of the compound of the invention and of the lipid, wherein the lipid encompasses a liposome forming lipid, a modified lipid (e.g., a PEG-lipid), and a non-liposome forming lipid. A skilled artisan will appreciate that the molar ratios of the essential constituents (i.e., the compound of the invention, the helper lipid, the structural lipid, and the modified lipid) within the LNP and within the composition of the invention are identical. Thus, the molar concentrations and molar ratios disclosed herein for example with respect to LNP also encompass the corresponding molar concentrations and molar ratios within the composition of the invention and vice versa.

In some embodiments, a molar concentration of the helper lipid within the nanoparticle is between 5 and 60 mol %, between 5 and 10 mol %, between 5 and 15 mol %, between 10 and 40 mol %, between 10 and 30 mol %, between 5 and 20 mol %, between 20 and 40 mol %, including any range between.

In some embodiments, a molar concentration of the structural lipid within the nanoparticle is between 5 and 60 mol %, between 20 and 60 mol %, between 10 and 50 mol %, between 20 and 50 mol %, between 5 and 40 mol %, between 20 and 40 mol %, between 30 and 40 mol %, including any range between.

In some embodiments, a molar concentration of the modified lipid (e.g., PEG-lipid) within the nanoparticle is between 0.5 and 10 mol %, between 0.1 and 10 mol %, between 0.1 and 0.5 mol %, between 0.5 and 1 mol %, between 1 and 5 mol %, between 0.5 and 2 mol %, between 5 and 10 mol %, between 5 and 7 mol %, between 7 and 10 mol %, including any range between.

In some embodiments, a molar ratio of the helper lipid and the modified-lipid ranges between 1:0.2 And 1:0.01, between 1:0.15 and 1:0.01, between 1:0.1 and 1:0.01, between 1:0.05 and 1:0.01, including any range in between.

In some embodiments, a molar ratio of the compound and the helper lipid ranges between 1:0.5 and 5:1, between 1:0.5 and 4:1, between 1:0.5. and 3:1, between 1:0.5 and 2:1, between 1:0.5 and 1:1, between 1:0.1 and 5:1, 1:1 and 1:2, between 1:1 and 1:5, between 1:0.25. and 5:1, between 1:0.5 and 2:1, including any range in between.

In some embodiments, a molar ratio of the structural lipid and the modified-lipid ranges between 200:1 and 2:1, between 100:1 and 5:1, between 100:1 and 10:1, between 100:1 and 30:1 between 100:1 and 50:1, between 100:1 and 70:1, between 100:1 and 90:1, between 100:1 and 100:3, between 100:1 and 20:1, between 150:1 and 20:1, between 200:1 and 50:1, between 200:1 and 10:1, including any range in between.

In some embodiments, a weight ratio between the compound of the invention (or the total lipid content, referring to the total amount of the compound of the invention and of the lipid(s) within the nanoparticle and/or within the composition comprising thereof) and the polynucleic acid within the nanoparticle (or within a composition comprising thereof) is between 0.001:1 and 10:1, between 0.001:1 and 0.1:1, between 0.1:1 and 1:1, between 1:1 and 10:1, including any range between.

In some embodiments, a N:P ratio within the lipid nanoparticle or within the composition of the invention ranges between 3 and 20, between 3 and 5, between 4 and 8, between 6 and 8, between 8 and 10, between 10 and 12, between 10 and 20, between 8 and 20, between 8 and 15, and between 12 and 14, including any range in between. The term “N:P ratio” refers to a ratio between N atoms of the compound of the invention and P atoms of the polynucleotide within the lipid nanoparticles or within the composition of the invention.

In some embodiments, the N:P ratio is about 12. In some embodiments, the N:P ratio within the lipid nanoparticle or within the composition of the invention ranges between 3 and 12.

In one embodiment, the nanoparticles within the composition are characterized by an average particle size of less than 500 nm to facilitate its entrance through the extracellular matrix to a cell. In one embodiment, the carrier is characterized by an average particle size of less than 300 nm in diameter to facilitate its entrance through the extracellular matrix to a cell.

In one embodiment, the nanoparticles within the composition are characterized by an average particle size of less than 300 nm, less than 250 nm, less than 200 nm, less than 150 nm, less than 100 nm, including any range between.

In another embodiment, the nanoparticles within the composition are characterized by an average particle size of between 50 and 300 nm, between 50 and 250 nm, between 50 and 200 nm, between 100 and 300 nm, between 50 and 100 nm, between 100 and 300 nm, including any range between. In another embodiment, the nanoparticles are characterized by an average particle size as disclosed herein and are further characterized by size distribution (polydispersity index, PDI) of between 0.05 and 0.4, between 0.05 and about 0.3, between about 0.1 and about 0.3, about 0.1, about 0.2, about 0.3, including any range between. In another embodiment, the average particle size and/or PDI is measured by Dynamic Light Scattering.

In another embodiment, the nanoparticle is characterized by a negative zeta potential or a positive zeta potential (e.g., measured at a pH about 7, e.g., between about 6.5 and 7.5). In some embodiments, the nanoparticle is characterized by a zeta potential ranging between −40 and +40 mV, including any range between. In some embodiments, the nanoparticle is characterized by a zeta potential ranging between −20 and +20 mV, including any range between. In some embodiments, the nanoparticle is characterized by a negative zeta potential ranging between −0.1 and −40 mV, including any range between. In some embodiments, the nanoparticle is characterized by a positive zeta potential ranging between +0.1 and +40 mV, including any range between. In some embodiments, the nanoparticle is characterized by a neutral zeta potential under physiological pH (such as a pH between 6.5 and 7.5 including any range between).

In some embodiments, the nanoparticle is stable for a time period ranging between 1 day and 1 year, or more, including any range between. In some embodiments, the term “stable” refers to physical and chemical stability of the nanoparticle (such as being substantially devoid of phase separation, agglomeration, disintegration, and/or substantially retaining the initial loading of the active agent) under appropriate storage conditions. In some embodiments, the term “stable” refers to physical and chemical stability of the nanoparticle within an aqueous solution (e.g., dispersion stability).

In some embodiments, the term “polynucleic acid” and the term “polynucleotide” are used herein interchangeably. In some embodiments, the polynucleotide comprises 60 to 15000 nucleobases, 15000 to 10000, 10000 to 4700, 200 to 5000 nucleobases, 300 to 5000 nucleobases, 400 to 5000 nucleobases, 400 to 2500 nucleobases, 200 to 3000 nucleobases, 400 to 2000 nucleobases, 400 to 1000 nucleobases, including any range between.

In some embodiments, the polynucleotide comprises at least 20 nucleobases, at least 250 nucleobases, at least 300 nucleobases, at least 350 nucleobases, at least 400 nucleobases, at least 450 nucleobases, at least 475 nucleobases, or at least 500 nucleobases. Each possibility represents a separate embodiment of the invention.

In some embodiments, the polynucleotide comprises 500 nucleobases at most, 750 nucleobases at most, 1,000 nucleobases at most, 1,250 nucleobases at most, 1,750 nucleobases at most, 2,500 nucleobases at most, 3000 nucleobases at most, 4000 nucleobases at most, or 5000 nucleobases at most. Each possibility represents a separate embodiment of the invention.

In some embodiments, the polynucleotide comprises a plurality of polynucleotide types. In some embodiments, the nanoparticle comprises a plurality of polynucleotide types. In some embodiments, the composition comprises a plurality of nanoparticle types, each type of nanoparticle comprises a specific polynucleotide.

In some embodiments, a specific polynucleotide comprises a plurality of polynucleotide molecules harboring the same or an identical nucleic acid sequence. In some embodiments, a specific polynucleotide comprises a plurality of polynucleotide molecules harboring essentially the same nucleic acid sequence.

As used herein, the term “plurality” encompasses any integer equal to or greater than 2. In some embodiments, a plurality comprises at least 2, at least 3, at least 4, at least 5, at least 6, at least 7, at least 8, at least 9, or at least 10, or any value and range therebetween. Each possibility represents a separate embodiment of the invention.

As used herein, the term “polynucleotide types” refers to a plurality of polynucleotides each of which comprises a nucleic acid sequence differing from any one of the other polynucleotides of the plurality of polynucleotides by at least 1 nucleobase, at least 1 nucleobase, at least 1 nucleobase, at least 1 nucleobase, at least 1 nucleobase, or at least 10 nucleobases, or any value and range therebetween. Each possibility represents a separate embodiment of the invention.

In some embodiments, a polynucleotide comprises RNA, DNA, a synthetic analog of RNA, circular RNA (circRNA), a synthetic analog of DNA, DNA/RNA hybrid, or any combination thereof. In some embodiments, a nanoparticle of the invention comprises a polynucleotide selected from: RNA, DNA, a synthetic analog of RNA, a synthetic analog of DNA, DNA/RNA hybrid, or any combination thereof.

In some embodiments, the polynucleotide comprises or consists of RNA. The polynucleotide comprises or consists of a messenger RNA (mRNA). “Messenger RNA” (mRNA) refers to any polynucleotide that encodes a (at least one) polypeptide (a naturally occurring, non-naturally occurring, or modified polymer of amino acids) and can be translated to produce the encoded polypeptide in vitro, in vivo, in situ or ex vivo. The basic components of an mRNA molecule typically include at least one coding region, a 5′ untranslated region (UTR), a 3′ UTR, a 5′ cap and a poly-A tail. Polynucleotides may function as mRNA but can be distinguished from wild-type mRNA in their functional and/or structural design features which serve to overcome existing problems of effective polypeptide expression using nucleic-acid based therapeutics.

The mRNA, as provided herein, comprises at least one (one or more) ribonucleic acid (RNA) polynucleotide having an open reading frame encoding at least one polypeptide of interest. In some embodiments, an RNA polynucleotide of an mRNA encodes 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5- 6, 6-10, 6-9, 6-8, 6-7, 7-10, 7-9, 7-8, 8-10, 8-9 or 9-10 polypeptides. In some embodiments, an RNA polynucleotide of an mRNA encodes at least 10, 20, 30, 40, 50, 60, 70, 80, 90 or 100 polypeptides. In some embodiments, an RNA polynucleotide of an mRNA encodes at least 100 or at least 200 polypeptides.

In some embodiments, the nucleic acids are therapeutic mRNAs. As used herein, the term “therapeutic mRNA” refers to an mRNA that encodes a therapeutic protein. Therapeutic proteins mediate a variety of effects in a host cell or a subject in order to treat a disease or ameliorate the signs and symptoms of a disease. For example, a therapeutic protein can replace a protein that is deficient or abnormal, augment the function of an endogenous protein, provide a novel function to a cell (e.g., inhibit or activate an endogenous cellular activity, or act as a delivery agent for another therapeutic compound (e.g., an antibody-drug conjugate). Therapeutic mRNA may be useful for the treatment of the following diseases and conditions: bacterial infections, viral infections, parasitic infections, cell proliferation disorders, genetic disorders, and autoimmune disorders.

Thus, the structures of the invention can be used as therapeutic or prophylactic agents. They are provided for use in medicine. For example, the mRNA of the structures described herein can be administered to a subject, wherein the polynucleotides are translated in vivo to produce a therapeutic peptide.

In some embodiments, the polynucleotide comprises an inhibitory nucleic acid. In some embodiments, the polynucleotide comprises an antisense oligonucleotide.

As used herein, an “antisense oligonucleotide” refers to a nucleic acid sequence that is reversed and complementary to a DNA or RNA sequence.

As referred to herein, a “reversed and complementary nucleic acid sequence” is a nucleic acid sequence capable of hybridizing with another nucleic acid sequence comprised of complementary nucleotide bases. By “hybridize” is meant pair to form a double-stranded molecule between complementary nucleotide bases (e.g., adenine (A) forms a base pair with thymine (T) (or uracil (U) in the case of RNA), and guanine (G) forms a base pair with cytosine (C)) under suitable conditions of stringency. (Sec, e.g., Wahl, G. M., and S. L. Berger (1987) Methods Enzymol. 152:399; Kimmel, A. R. (1987) Methods Enzymol. 152:507). For the purposes of the present methods, the inhibitory nucleic acid need not be complementary to the entire sequence, only enough of it to provide specific inhibition; for example, in some embodiments the sequence is 100% complementary to at least nucleotides (nts) 2-7 or 2-8 at the 5′ end of the microRNA itself (e.g., the ‘seed sequence’), e.g., nts 2-7 or 20.

In some embodiments of the inhibitory nucleic acid has one or more chemical modifications to the backbone or side chains. In some embodiments, the inhibitory nucleic acid has at least one locked nucleotide, and/or has a phosphorothioate backbone.

Non-limiting examples of inhibitory nucleic acids useful according to the herein disclosed invention include, but are not limited to: antisense oligonucleotides, ribozymes, external guide sequence (EGS) oligonucleotides, siRNA compounds, single- or double-stranded RNA interference (RNAi) compounds such as siRNA compounds, modified bases/locked nucleic acids (LNAs), antagomirs, peptide nucleic acids (PNAs), ribozymes (catalytic RNA molecules capable to cut other specific sequences of RNA molecules) and other oligomeric compounds or oligonucleotide mimetics which hybridize to at least a portion of the target nucleic acid and modulate its function. In some embodiments, the inhibitory nucleic acids include antisense RNA, antisense DNA, chimeric antisense oligonucleotides, antisense oligonucleotides comprising modified linkages, interference RNA (RNAi), short interfering RNA (siRNA); a micro-RNA (miRNA); a small, temporal RNA (stRNA); or a short, hairpin RNA (shRNA); small RNA-induced gene activation (RNAa); small activating RNAs (saRNAs), or combinations thereof.

In some embodiments, the inhibitory nucleic acid is an RNA interfering molecule (RNAi). In some embodiments, the RNAi is or comprises double stranded RNA (dsRNA).

As used herein “an interfering RNA” refers to any double stranded or single stranded RNA sequence, capable—either directly or indirectly (i.e., upon conversion)—of inhibiting or down regulating gene expression by mediating RNA interference. Interfering RNA includes but is not limited to small interfering RNA (“siRNA”) and small hairpin RNA (“shRNA”). “RNA interference” refers to the selective degradation of a sequence-compatible messenger RNA transcript.

In some embodiments, the polynucleotide is chemically modified. In some embodiments, the chemical modification is a modification of a backbone of the polynucleotide. In some embodiments, the chemical modification is a modification of a sugar of the polynucleotide. In some embodiments, the chemical modification is a modification of a nucleobase of the polynucleotide. In some embodiments, the chemical modification increases stability of the polynucleotide in a cell. In some embodiments, the chemical modification increases stability of the polynucleotide in vivo. In some embodiments, the chemical modification increases the stability of the polynucleotide in vitro, such as, in the open air, field, on a surface exposed to air, etc. In some embodiments, the chemical modification increases the polynucleotide's ability to induce silencing of a target gene or sequence, including, but not limited to an RNA molecule derived from a pathogen or an RNA derived from a plant cell, as described herein. In some embodiments, the chemical modification is selected from: a phosphate-ribose backbone, a phosphate-deoxyribose backbone, a phosphorothioate-deoxyribose backbone, a 2′-O-methyl-phosphorothioate backbone, a phosphorodiamidate morpholino backbone, a peptide nucleic acid backbone, a 2-methoxyethyl phosphorothioate backbone, a constrained ethyl backbone, an alternating locked nucleic acid backbone, a phosphorothioate backbone, N3′-P5′ phosphoroamidates, 2′-deoxy-2′-fluoro-β-d-arabino nucleic acid, cyclohexene nucleic acid backbone nucleic acid, tricyclo-DNA (tcDNA) nucleic acid backbone, ligand-conjugated antisense, and a combination thereof.

In some embodiments, the carrier (e.g., a lipid nanoparticle) is prepared by combining an aqueous phase optionally comprising an active agent, and an organic phase comprising one or more lipid components and the compound of the invention. The selection of specific lipids (such as cationic lipids, non-cationic lipids, sterol(s) and/or PEG-modified lipids) which comprise the lipid nanoparticle, as well as the relative molar ratio of such lipids to each other and/or a molar ratio between the lipid(s) and the compound of the invention, is based upon the characteristics of the selected lipid(s), and the characteristics of the agents to be delivered. Additional considerations include, for example, the saturation of the alkyl chain, as well as the size, Tm, charge, pH, pKa, fusogenicity and toxicity of the selected lipid(s).

In another aspect, there is provided a pharmaceutical composition comprising the lipid nanoparticles of the invention and a pharmaceutically acceptable carrier. In some embodiments, the pharmaceutically acceptable carrier is also referred to as an excipient or adjuvant. As used herein, the term “carrier,” “excipient,” or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers, and diluents useful in the present compositions include distilled water, physiological saline, Hartmann solution, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

In some embodiments, the pharmaceutical composition is formulated for systemic administration. In some embodiments, the pharmaceutical composition is formulated for administration to a subject. In some embodiments, the subject is a mammal. In some embodiments, the mammal is a human. In some embodiments, the mammal is a laboratory animal. Examples of laboratory animals include, but are not limited to, mice, rats, rabbits, hamsters, dogs, cats, and monkeys. In some embodiments, the mammal is a mouse or rat. In some embodiments, the subject is in need of the composition. In some embodiments, the subject is in need of treatment. In some embodiments, the subject is a volunteer for a diagnostic method. In some embodiments, the subject is in need of diagnosis.

In some embodiments, the pharmaceutical composition is for use in a therapeutic method. In some embodiments, a therapeutic method is a method of treatment. In some embodiments, the pharmaceutical composition is for use in a diagnostic method. In some embodiments, the method comprises administering the composition of the invention to a subject. In some embodiments, the pharmaceutical composition is for use in treatment or prevention of a disease or condition in humans and other mammals. The active therapeutic agents of the invention include the nanoparticles, or polypeptides translated from the polynucleotides contained in the nanoparticles.

As used herein, the terms “administering,” “administration,” and like terms refer to any method which, in sound medical practice, delivers a composition containing an active agent to a subject in such a manner as to provide a therapeutic effect. One aspect of the present subject matter provides for intravenous administration of a therapeutically effective amount of a composition of the present subject matter to a patient in need thereof. Other suitable routes of administration can include parenteral, intravenous, subcutaneous, oral, intramuscular, intrathecal, inhaled, intracerebroventricular, intravitreal, transdermal, or intraperitoneal.

The dosage administered will be dependent upon the age, health, and weight of the recipient, kind of concurrent treatment, if any, frequency of treatment, and the nature of the effect desired.

By another aspect, there is provided a method of treating a subject in need thereof, the method comprising administering to the subject a therapeutic composition of the invention.

In some embodiments, there is provided a method for delivering an active agent into a cell of a subject, comprising contacting the cell with the nanoparticle(s) of the invention, wherein each nanoparticle comprises the active agent (e.g. wherein the active agent is encapsulated within the nanoparticle). In some embodiments, the active agent is a described hereinabove.). In some embodiments, the active agent is cell impermeable. Cell delivery (i.e. intracellular delivery) can be determined by quantifying the amount of the active agent inside the cell (e.g. by fluorescent labeling of the compound, or in the case of a polynucleotide, by determining the expression level of the polynucleotide).

In some embodiments, delivering is so as to obtain an increased concentration of the active agent within the cell, as compared to a control formulation comprising the same active agent and lipofectamine as the cell internalizing agent instead of the lipid nanoparticles of the invention. In some embodiments, increased concentration is a therapeutically effective concentration. In some embodiments, increased concentration is referred to at least 10 times, at least 50 times, at least 100 times, at least 200 times greater concentration, as compared to the control.

In some embodiments, the cell is a tissue cell. In some embodiments, the method is for delivering the active agent to a tissue of the subject. In some embodiments, delivering is to obtain a therapeutically effective concentration of the active agent within the tissue. In some embodiments, delivering is to obtain an increased amount of the active agent within the tissue, wherein increased is as compared to the control, as disclosed herein.

### Definitions

As used herein, the term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the term “substantially” refers to at least 85%, at least 90%, at least 92%, at least 95%, at least 96%, at least 97%, at least 98%, at least 99%, or between 60 and 99.9%, between 70 and 80%, between 70 and 90%, between 80 and 90%, between 90 and 95%, between 95 and 99.9%, including any range or value therebetween.

As used herein, the term “substituent” encompasses hydrogen, halogen, —NO₂, —CN, —OH, oxo, imino, —CONH₂, —CONR′₂, —CNNR′₂, —CSNR′₂, —CONH—OH, —CONH—NH₂, —NHCOR, —NHCSR, —NHCNR, —NC(═O) OR, —NC(═O)NR′, —NC(═S) OR′, —NC(═S)NR′, —SO₂R′, —SOR′, —SR′, —SO₂OR′, —SO₂N(R′)₂, —NHNR′₂, —NNR′, C₁-C₆ haloalkyl, optionally substituted C₁-C₆ alkyl, —NH₂, —NR′₂, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′₂, C₁-C₆ alkyl-SR′, —CONH(C₁-C₆ alkyl), —CON(C₁-C₆ alkyl)₂, —CO₂H, —CO₂R′, —OCOR, —OCOR′, —OC(═O)OR′, —OC(═O)NR′, —OC(═S) OR′, —OC(═S)NR′, or a combination thereof; wherein each R′ independently represents hydrogen, or is selected from the group comprising optionally substituted C₁-C₁₀ alkyl, optionally substituted C₃-C₁₀ cycloalkyl, optionally substituted C₃-C₁₀ heterocyclyl, optionally substituted heteroaryl, optionally substituted aryl, hydroxy, amino, —NH₂, —NR′₂—NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′₂, C₁-C₆ alkyl —SR′, or a combination thereof.

As used herein, the term “alkyl” describes an aliphatic hydrocarbon including straight chain and branched chain groups. The term “alkyl”, as used herein, also encompasses saturated or unsaturated hydrocarbon, hence this term further encompasses alkenyl and alkynyl.

The term “alkenyl” describes an unsaturated alkyl, as defined herein, having at least two carbon atoms and at least one carbon-carbon double bond. The alkenyl may be substituted or unsubstituted by one or more substituents, as described hereinabove.

The term “alkynyl”, as defined herein, is an unsaturated alkyl having at least two carbon atoms and at least one carbon-carbon triple bond. The alkynyl may be substituted or unsubstituted by one or more substituents, as described hereinabove.

The term “cycloalkyl” describes an all-carbon monocyclic or fused ring (i.e., rings which share an adjacent pair of carbon atoms) group where one or more of the rings does not have a completely conjugated pi-electron system. The cycloalkyl group may be substituted or unsubstituted, as indicated herein.

The term “aryl” describes an all-carbon monocyclic or fused-ring polycyclic (i.e., rings which share adjacent pairs of carbon atoms) groups having a completely conjugated pi-electron system. The aryl group may be substituted or unsubstituted, as indicated herein.

The term “alkoxy” describes both an O-alkyl and an —O-cycloalkyl group, as defined herein. The term “aryloxy” describes an —O-aryl, as defined herein.

Each of the alkyl, cycloalkyl and aryl groups in the general formulas herein may be substituted by one or more substituents, whereby each substituent group can independently be, for example, halide, alkyl, alkoxy, cycloalkyl, nitro, amino, hydroxyl, thiol, thioalkoxy, carboxy, amide, aryl and aryloxy, depending on the substituted group and its position in the molecule. Additional substituents are also contemplated.

The term “halide”, “halogen” or “halo” describes fluorine, chlorine, bromine, or iodine. The term “haloalkyl” describes an alkyl group as defined herein, further substituted by one or more halide(s). The term “haloalkoxy” describes an alkoxy group as defined herein, further substituted by one or more halide(s). The term “hydroxyl” or “hydroxy” describes a —OH group. The term “mercapto” or “thiol” describes a —SH group. The term “thioalkoxy” describes both an —S-alkyl group, and a —S-cycloalkyl group, as defined herein. The term “thioaryloxy” describes both an —S-aryl and a —S-heteroaryl group, as defined herein. The term “amino” describes a —NR′R″ group, or a salt thereof, with R′ and R″ as described herein.

The term “heterocyclyl” describes a monocyclic or fused ring group having in the ring(s) one or more atoms such as nitrogen, oxygen, and sulfur. The rings may also have one or more double bonds. However, the rings do not have a completely conjugated pi-electron system. Representative examples are piperidine, piperazine, tetrahydrofuran, tetrahydropyran, morpholino and the like.

The term “carboxy” describes a —C(O)OR′ group, or a carboxylate salt thereof, where R′ is hydrogen, alkyl, cycloalkyl, alkenyl, aryl, heteroaryl (bonded through a ring carbon) or heterocyclyl (bonded through a ring carbon) as defined herein. or “carboxylate”

The term “carbonyl” describes a —C(O)R′ group, where R′ is as defined hereinabove. The above-terms also encompass thio-derivatives thereof (thiocarboxy and thiocarbonyl).

The term “thiocarbonyl” describes a —C(S)R′ group, where R′ is as defined hereinabove. A “thiocarboxy” group describes a —C(S)OR′ group, where R′ is as defined herein. A “sulfinyl” group describes an —S(O)R′ group, where R′ is as defined herein. A “sulfonyl” or “sulfonate” group describes an —S(O)2R′ group, where R′ is as defined herein.

A “carbamyl” or “carbamate” group describes an —OC(O)NR′R″ group, where R′ is as defined herein and R″ is as defined for R′. A “nitro” group refers to a —NO2 group. The term “amide” as used herein encompasses C-amide and N-amide. The term “C-amide” describes a —C(O)NR′R″ end group or a —C(O)NR′-linking group, as these phrases are defined hereinabove, where R′ and R″ are as defined herein. The term “N-amide” describes a —NR″C(O)R′ end group or a —NR′C(O)— linking group, as these phrases are defined hereinabove, where R′ and R″ are as defined herein.

A “cyano” or “nitrile” group refers to a —CN group. The term “azo” or “diazo” describes an —N═NR′ end group or an —N═N— linking group, as these phrases are defined hereinabove, with R′ as defined hereinabove. The term “guanidine” describes a —R′NC(N)NR″R′″ end group or a —R′NC(N) NR″— linking group, as these phrases are defined hereinabove, where R′, R″ and R′″ are as defined herein. As used herein, the term “azide” refers to a —N3 group. The term “sulfonamide” refers to a —S(O)2NR′R″ group, with R′ and R″ as defined herein.

The term “phosphonyl” or “phosphonate” describes an —OP(O)—(OR′)2 group, with R′ as defined hereinabove. The term “phosphinyl” describes a —PR′R″ group, with R′ and R″ as defined hereinabove. The term “alkylaryl” describes an alkyl, as defined herein, which substituted by an aryl, as described herein. An exemplary alkylaryl is benzyl.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. As used herein, the term “heteroaryl” refers to an aromatic ring in which at least one atom forming the aromatic ring is a heteroatom. Heteroaryl rings can be foamed by three, four, five, six, seven, eight, nine and more than nine atoms. Heteroaryl groups can be optionally substituted. Examples of heteroaryl groups include, but are not limited to, aromatic C₃₋₈ heterocyclic groups containing one oxygen or sulfur atom, or two oxygen atoms, or two sulfur atoms or up to four nitrogen atoms, or a combination of one oxygen or sulfur atom and up to two nitrogen atoms, and their substituted as well as benzo- and pyrido-fused derivatives, for example, connected via one of the ring-forming carbon atoms. In certain embodiments, heteroaryl is selected from among oxazolyl, isoxazolyl, oxadiazolyl, thiazolyl, isothiazolyl, pyridinyl, pyridazinyl, pyrimidinal, pyrazinyl, indolyl, benzimidazolyl, quinolinyl, isoquinolinyl, quinazolinyl or quinoxalinyl.

In some embodiments, a heteroaryl group is selected from among pyrrolyl, furanyl (furyl), thiophenyl (thienyl), imidazolyl, pyrazolyl, 1,2,3-triazolyl, 1,2,4-triazolyl, 1,3-oxazolyl (oxazolyl), 1,2-oxazolyl (isoxazolyl), oxadiazolyl, 1,3-thiazolyl (thiazolyl), 1,2-thiazolyl (isothiazolyl), tetrazolyl, pyridinyl (pyridyl)pyridazinyl, pyrimidinyl, pyrazinyl, 1,2,3-triazinyl, 1,2,4-triazinyl, 1,3,5-triazinyl, 1,2,4,5-tetrazinyl, indazolyl, indolyl, benzothiophenyl, benzofuranyl, benzothiazolyl, benzimidazolyl, benzodioxolyl, acridinyl, quinolinyl, isoquinolinyl, quinazolinyl, quinoxalinyl, phthalazinyl, thienothiophenyl, 1,8-naphthyridinyl, other naphthyridinyls, pteridinyl or phenothiazinyl. Where the heteroaryl group includes more than one ring, each additional ring is the saturated form (perhydro form) or the partially unsaturated form (e.g., the dihydro form or tetrahydro form) or the maximally unsaturated (nonaromatic) form. The term heteroaryl thus includes bicyclic radicals in which the two rings are aromatic and bicyclic radicals in which only one ring is aromatic. Such examples of heteroaryl are include 3H-indolinyl, 2 (1H)-quinolinonyl, 4-oxo-1,4-dihydroquinolinyl, 2H-1-oxoisoquinolyl, 1,2-dihydroquinolinyl, (2H) quinolinyl N-oxide, 3,4-dihydroquinolinyl, 1,2-dihydroisoquinolinyl, 3,4-dihydro-isoquinolinyl, chromonyl, 3,4-dihydroiso-quinoxalinyl, 4-(3H) quinazolinonyl, 4H-chromenyl, 4-chromanonyl, oxindolyl, 1,2,3,4-tetrahydroisoquinolinyl, 1,2,3,4-tetrahydro-quinolinyl, 1H-2,3-dihydroisoindolyl, 2,3-dihydrobenzo[f]isoindolyl, 1,2,3,4-tetrahydrobenzo-[g]isoquinolinyl, 1,2,3,4-tetrahydro-benzo[g]isoquinolinyl, chromanyl, isochromanonyl, 2,3-dihydrochromonyl, 1,4-benzo-dioxanyl, 1,2,3,4-tetrahydro-quinoxalinyl, 5,6-dihydro-quinolyl, 5,6-dihydroiso-quinolyl, 5,6-dihydroquinoxalinyl, 5,6-dihydroquinazolinyl, 4,5-dihydro-1H-benzimidazolyl, 4,5-dihydro-benzoxazolyl, 1,4-naphthoquinolyl, 5,6,7,8-tetrahydro-quinolinyl, 5,6,7,8-tetrahydro-isoquinolyl, 5,6,7,8-tetrahydroquinoxalinyl, 5,6,7,8-tetrahydroquinazolyl, 4,5,6,7-tetrahydro-1H-benzimidazolyl, 4,5,6,7-tetrahydro-benzoxazolyl, 1H-4-oxa-1,5-diaza-naphthalen-2-onyl, 1,3-dihydroimidizolo-[4,5]-pyridin-2-onyl, 2,3-dihydro-1,4-dinaphtho-quinonyl, 2,3-dihydro-1H-pyrrol[3,4-b]quinolinyl, 1,2,3,4-tetrahydrobenzo[b]-[1,7]naphthyridinyl, 1,2,3,4-tetra-hydrobenz[b][1,6]-naphthyridinyl, 1,2,3,4-tetrahydro-9H-pyrido[3,4-b]indolyl, 1,2,3,4-tetrahydro-9H-pyrido[4,3-b]indolyl, 2,3-dihydro-1H-pyrrolo-[3,4-b]indolyl, 1H-2,3,4,5-tetrahydro-azepino[3,4-b]indolyl, 1H-2,3,4,5-tetrahydroazepino-[4,3-b]indolyl, 1H-2,3,4,5-tetrahydro-azepino[4,5-b]indolyl, 5,6,7,8-tetrahydro[1,7]napthyridinyl, 1,2,3,4-tetrahydro-[2,7]-naphthyridyl, 2,3-dihydro[1,4]dioxino[2,3-b]pyridyl, 2,3-dihydro[1,4]-dioxino[2,3-b]pyridyl, 3,4-dihydro-2H-1-oxa[4,6]diazanaphthalenyl, 4,5,6,7-tetrahydro-3H-imidazo-[4,5-c]pyridyl, 6,7-dihydro[5,8]diazanaphthalenyl, 1,2,3,4-tetrahydro[1,5]-napthyridinyl, 1,2,3,4-tetrahydro[1,6]napthyridinyl, 1,2,3,4-tetrahydro[1,7]napthyridinyl, 1,2,3,4-tetrahydro-[1,8]napthyridinyl or 1,2,3,4-tetrahydro[2,6]napthyridinyl. In some embodiments, heteroaryl groups are optionally substituted. In one embodiment, the one or more substituents are each independently selected from among halo, hydroxy, amino, cyano, nitro, alkylamido, acyl, C₁₋₆-alkyl, C₁₋₆-haloalkyl, C₁₋₆-hydroxyalkyl, C₁₋₆-aminoalkyl, C₁₋₆-alkylamino, alkylsulfenyl, alkylsulfinyl, alkylsulfonyl, sulfamoyl, or trifluoromethyl.

Examples of heteroaryl groups include, but are not limited to, unsubstituted and mono- or di-substituted derivatives of furan, benzofuran, thiophene, benzothiophene, pyrrole, pyridine, indole, oxazole, benzoxazole, isoxazole, benzisoxazole, thiazole, benzothiazole, isothiazole, imidazole, benzimidazole, pyrazole, indazole, tetrazole, quinoline, isoquinoline, pyridazine, pyrimidine, purine and pyrazine, furazan, 1,2,3-oxadiazole, 1,2,3-thiadiazole, 1,2,4-thiadiazole, triazole, benzotriazole, pteridine, phenoxazole, oxadiazole, benzopyrazole, quinolizine, cinnoline, phthalazine, quinazoline and quinoxaline. In some embodiments, the substituents are halo, hydroxy, cyano, O—C₁₋₆-alkyl, C₁₋₆-alkyl, hydroxy-C₁₋₆-alkyl, and amino-C₁₋₆-alkyl.

As used herein, the terms “halo” and “halide”, which are referred to herein interchangeably, describe an atom of a halogen, that is fluorine, chlorine, bromine, or iodine, also referred to herein as fluoride, chloride, bromide, and iodide.

### GENERAL

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description or claims, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological, and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization—A Laboratory Course Manual” CSHL Press (1996); all of which are incorporated by reference. Other general references are provided throughout this document.

### Example 1

Exemplary compounds of the invention have been synthesized according to synthetic schemes presented hereinbelow. The inventors successfully utilized exemplary compounds of the invention for the preparation of stable LNPs, which have been tested in a cell-based assay to explore its expression efficiency. Exemplary compounds of the invention (ionizable lipids) are presented hereinbelow. Vast majority of these compounds below have been tested and were found capable to form RNA encapsulating LNPs. Additional compounds/LNP formulations currently undergo screening in various in-vivo/in-vitro assays.

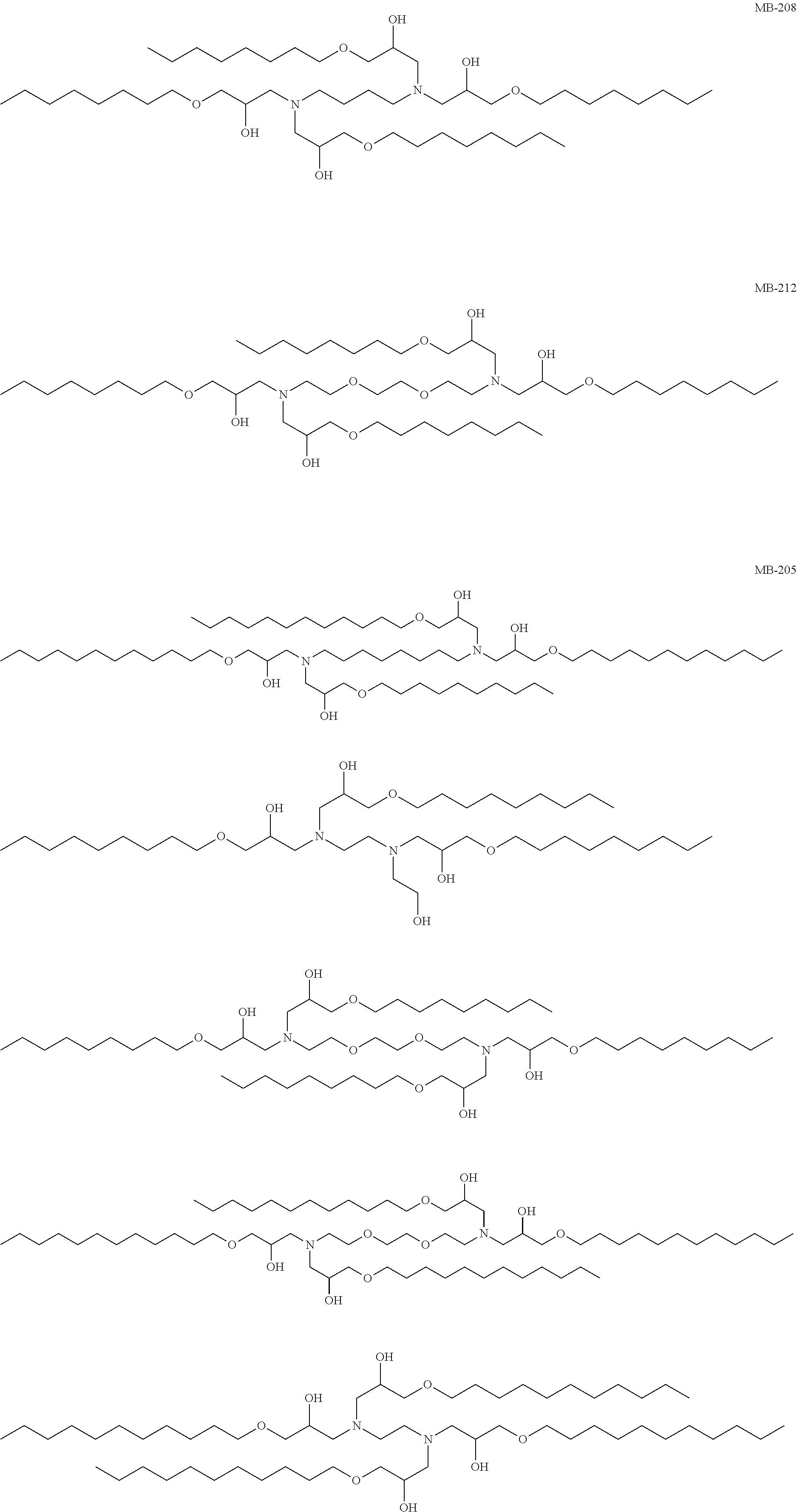

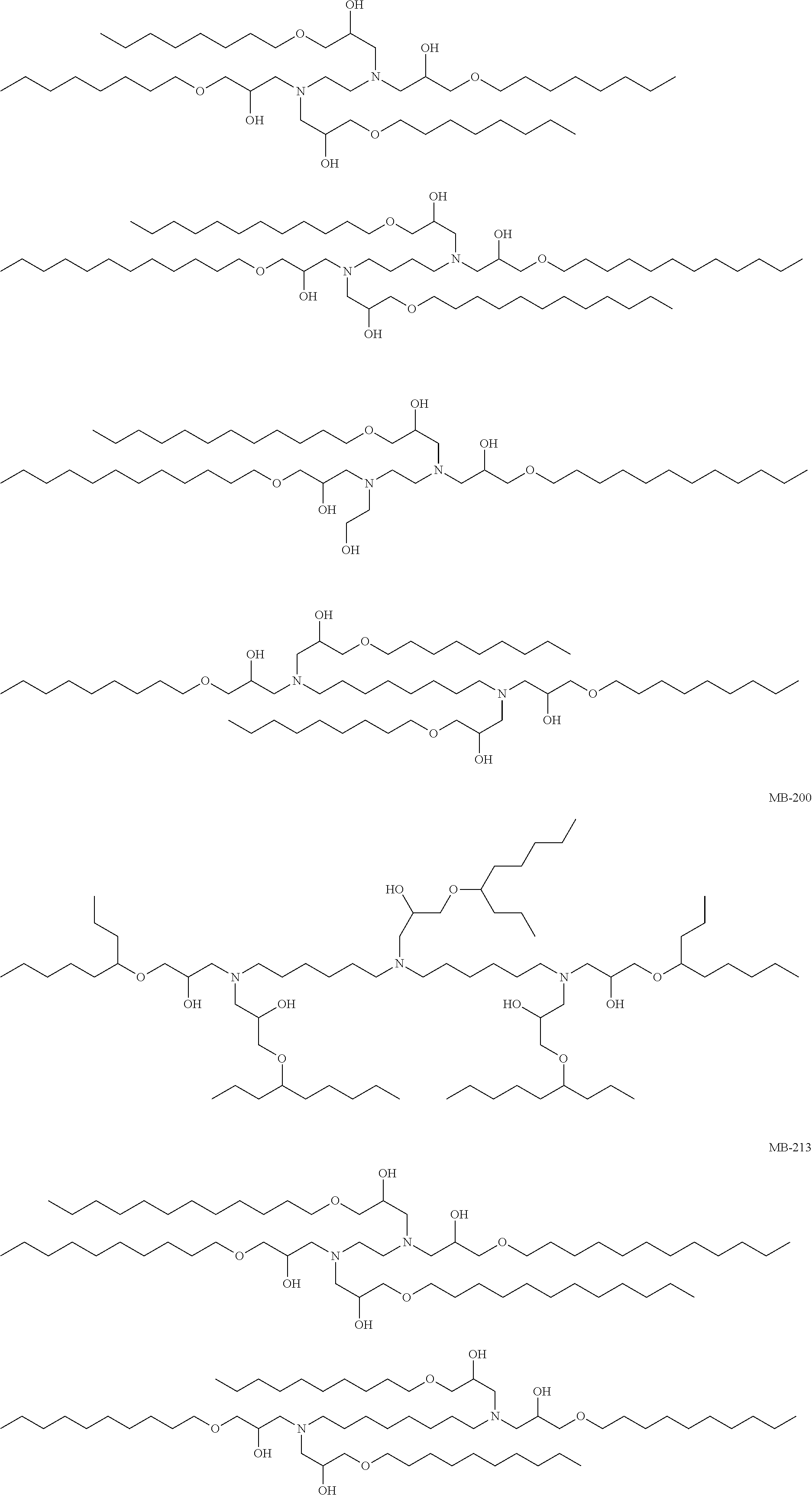

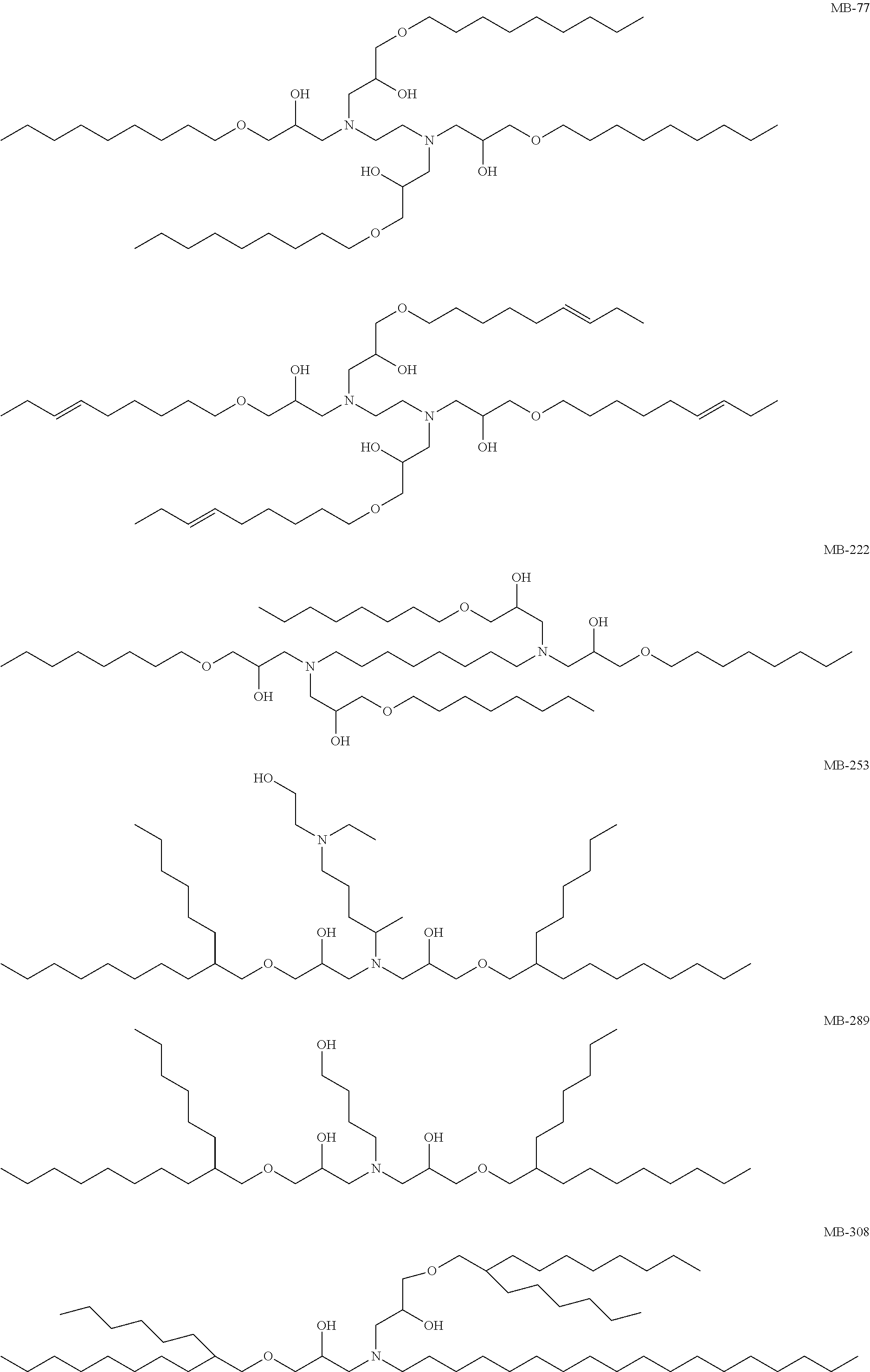

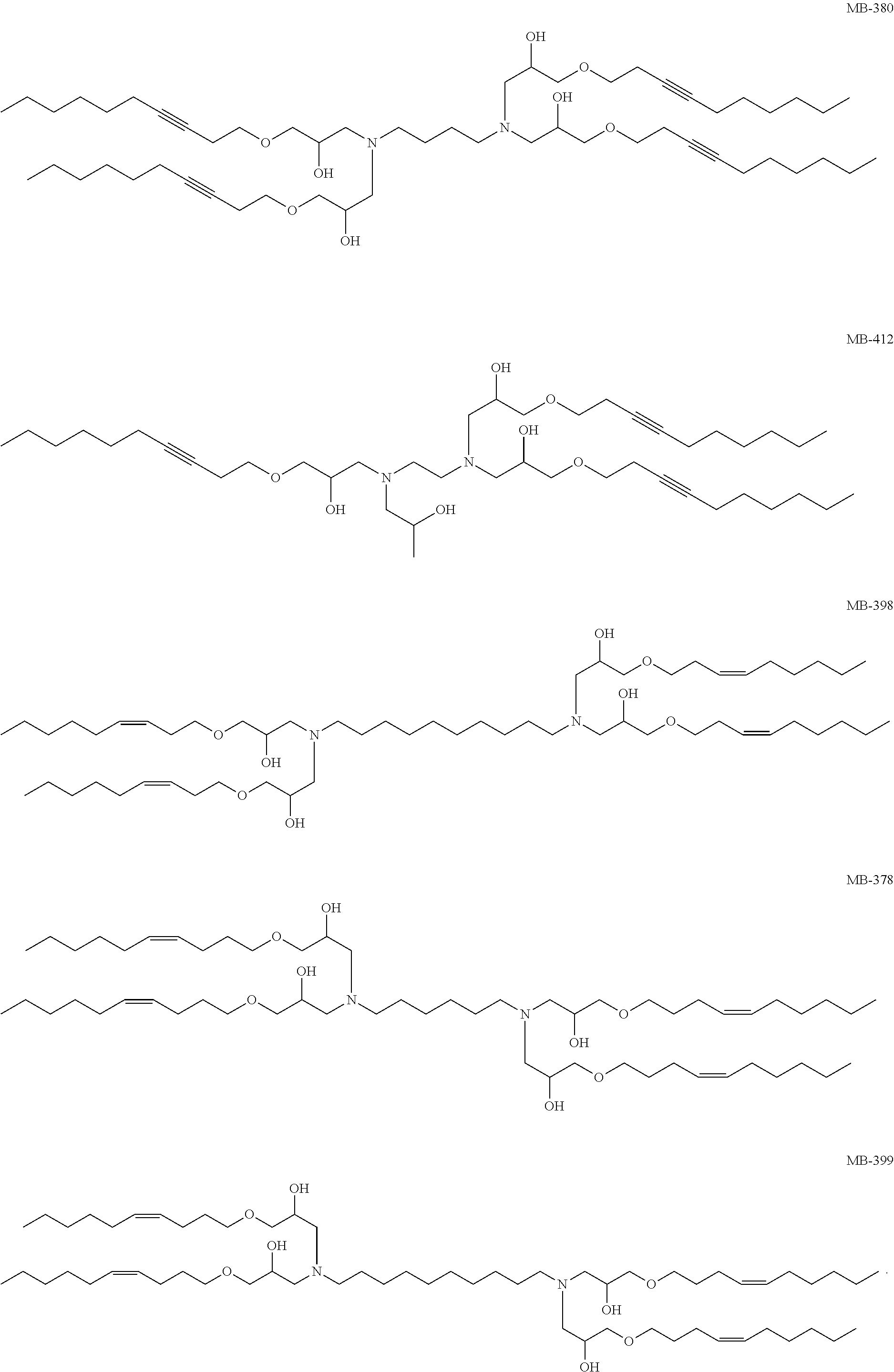

Some of the exemplary LNPs of invention have been characterized by superior cell penetration, as determined in cell-based studies. For example, LNPs containing MB-77, 208, 212, and 205 (exemplary compounds of the invention) exhibited superior internalization efficiency, as compared to a similar composition comprising Dlin-MC3-DMA as the ionizable lipid.

A general synthetic scheme for some of the exemplary compounds of the invention is presented herein. Other possible synthetic strategies are well-known to a skilled artisan.

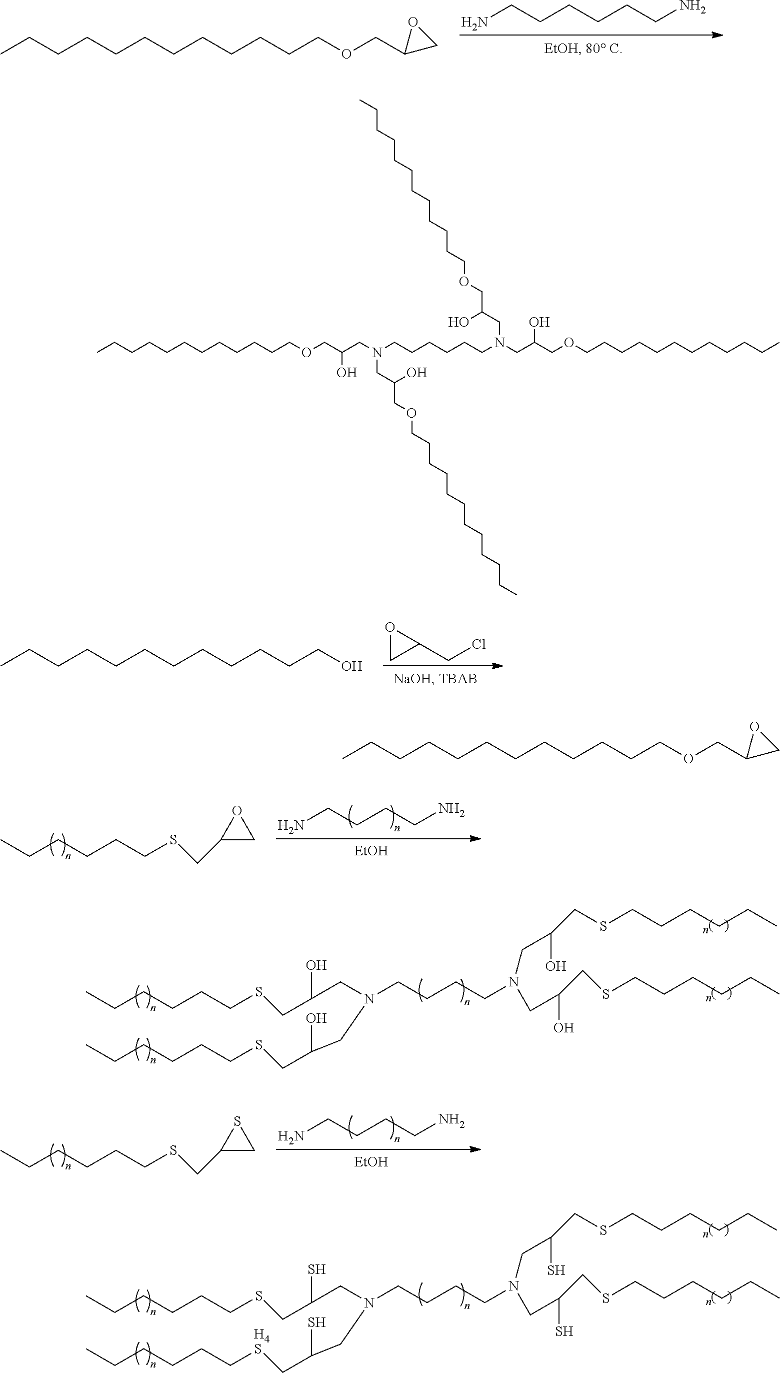

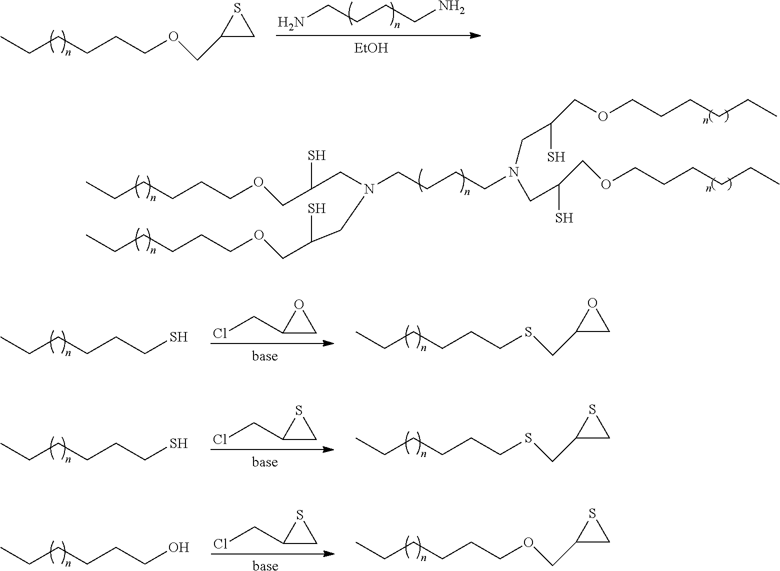

A composition of an exemplary LNP is as follows: a helper lipid (e.g., DOPE) about 5-15 mol %; a structural lipid (e.g., cholesterol) about 30-45 mol %; PEG-lipid (e.g., DMG-PEG2000) between 0.5 and 5 mol %; and a compound of the invention about 40-60 mol %. The LNPs prepared by the inventors have been characterized by an average particle size ranging between about 60 and about 300 nm.

The LNPs have been prepared as follows: lipids were weighed and solubilized in Ethanol (EtOH) at 55-60° C. mRNA was added to citrate buffer at pH of 5.0 (range 4.5-5.5). Mixing of lipids containing PEG-lipid, helper lipid, cholesterol, and Ionizable-lipid (compound) was done under microfluidic mixing or by EtOH injection of the lipids into the mRNA containing citrate buffer under constant mixing conditions. pH of the mixture was then elevated using PBS dilution and residual EtOH was removed prior to injection using dialysis such as FloatAlyzer or standard dialysis membranes using cutoff of 3 kDa, 8 kDa, 12-14 kDa or 100 kDa according to separation requirements.

The encapsulation efficiency has been calculated by Ribogreen assay, Quant-iT™ RiboGreen® RNA Reagent and Kit (Invitrogen) following manufacturer directions, where particles were diluted using Tris-EDTA buffer and supplemented with RiboGreen (ThermoFisher) diluted 1:2000 and read for fluorescence using plate reader (mRNA signal not encapsulated in LNPs). triton ×100 was diluted in TE and was added to each well and incubated for 10 minutes at 37° C. to allow perturbation of LNPs. fluorescence was measured again (mRNA signal of total mRNA in formulation). Encapsulation efficiency was calculated using % EE=100*(total−out)/total.

The inventors determined cell-penetration of the exemplary LNPs and intracellular delivery of mRNA.

More specifically, particles were incubated on cells for 16-24 hrs at 37° C. under 5% CO2 and expression levels were measured. Formulations were compared to Lipofectamine control and to MC3 control, as well as to untreated controls.

Additional exemplary formulations were examined, stable LNPs were obtained with an average particle size of between 60 and 210 nm and low PDI (between 0.1 and 0.3), results are summarized in table 1.

TABLE 1
formulation of exemplary LNPs

| | | | | | | Modified | | |
|---|---|---|---|---|---|---|---|---|
| | Ionizable | Helper | Helper | structural | structural | lipid (PEG- | Modified | |
| Ionizable lipid | lipid, % | lipid | lipid, % | lipid | lipid, % | lipid) | lipid, % | N:P |
| MB-222 | 50 | DOPE | 10 | cholesterol | 38.5 | DMG- | 1.5 | 8 |
| | | | | | | PEG2000 | | |
| MB-222 | 35 | DOTAP | 40 | cholesterol | 22.5 | DMG- | 2.5 | 8.4 |
| | | | | | | PEG2000 | | |
| MB-212 | 50 | DOPE | 10 | cholesterol | 38.5 | DMG- | 1.5 | 8 |
| | | | | | | PEG2000 | | |
| MB-212 | 20 | DOTAP | 40 | cholesterol | 38.5 | DMG- | 1.5 | 4.8 |
| | | | | | | PEG2000 | | |
| MB-209 | 50 | DOPE | 10 | cholesterol | 38.5 | DMG- | 1.5 | 8 |
| | | | | | | PEG2000 | | |

### Example 2

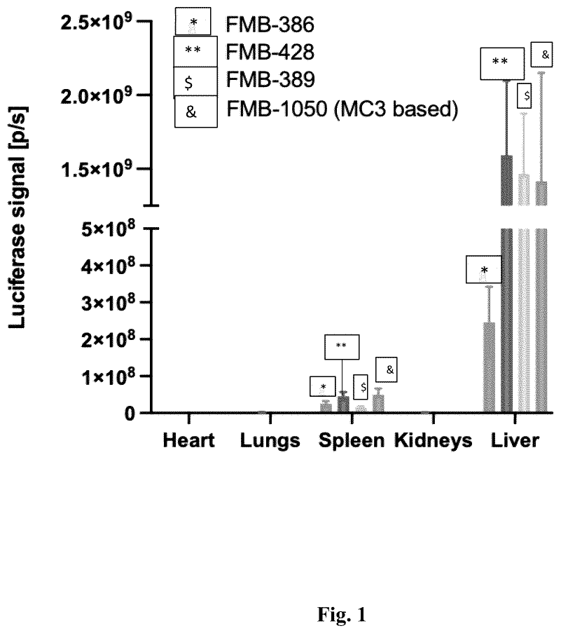

Some of the exemplary compositions of the invention have been characterized by enhanced specificity to lung cells, as determined in cell-based studies. For example, LNPs of the invention containing MB-212 or MB-222 as the ionizable lipid, exhibited enhanced specificity to the lung cells, as compared to a similar composition comprising Dlin-MC3-DMA as the ionizable lipid (FIG. 1). Surprisingly, the inventors found that exemplary LNPs containing between about 30 and about 50 of a helper lipid (e.g. DOTAP), between about 20 and about 50 of a structural lipid (cholesterol), and between about 0.5 and about 5 of a modified lipid (e.g. PEG-lipid), along with between about 15 and about 50% of the ionizable lipid (e.g. MB-212 or MB-222) resulted in enhanced lung specificity of the LNP, as evaluated by expression analysis in-vivo. Exemplary LNP compositions of the invention showed high lung specificity resulting in significantly enhanced expression of the encapsulated polynucleic acid (mRNA F-LUC) in the lung, as compared to a commercial control (FIG. 1). Furthermore, a specific LNP compositions of the invention (FMB-1143) comprising between about 15 and about 30% of the ionizable lipid exhibited superior lung specificity (see FIG. 1).

### Preparation of FMB-745

40% of DOTAP, 22.5% of cholesterol, 2.5% DMG-PEG2000 and 35% of a commercial ionizable lipid (Dlin-MC3-DMA) were solubilized in ethanol (EtOH) at 55-60° C. mRNA, F-LUC was added to citrate buffer at pH of 5.0 (range 4.5-5.5). Mixing of lipids was done under microfluidic mixing or by EtOH injection of the lipids into mRNA firefly luciferase (F-LUC) containing citrate buffer under constant mixing conditions. pH of the mixture was then elevated using PBS dilution and residual EtOH was removed prior to injection using dialysis prepared by the inventors have been characterized by an average particle size ranging between about 60 and about 130 nm.

### Preparation of FMB-748

40% of DOTAP, about 22% of cholesterol, about 2-2.5% DMG PEG2000 and 35% of ionizable MB-222 were solubilized in ethanol (EtOH) at 55-60° C. mRNA, F-LUC was added to citrate buffer at pH of 5.0 (range 4.5-5.5). Mixing of lipids was done under microfluidic mixing or by EtOH injection of the lipids into mRNA F-LUC containing citrate buffer under constant mixing conditions. pH of the mixture was then elevated using PBS dilution and residual EtOH was removed prior to injection using dialysis prepared by the inventors have been characterized by an average particle size ranging between about 60 and about 130 nm.

### Preparation of Lung-Specific Formulation (FMB-1143)

40% of DOTAP, about 38% of cholesterol, 1.5% DMG-PEG2000 and 20% of ionizable MB-212 were solubilized in ethanol (EtOH) at 55-60° C. mRNA, F-LUC was added to citrate buffer at pH of 5.0 (range 4.5-5.5). Mixing of lipids was done under microfluidic mixing or by EtOH injection of the lipids into the mRNA F-LUC containing citrate buffer under constant mixing conditions. pH of the mixture was then elevated using PBS dilution and residual EtOH was removed prior to injection using dialysis prepared by the inventors have been characterized by an average particle size ranging between about 60 and about 130 nm.

The inventors determined the expression distribution of the exemplary LNPs in vivo by assessing expression of the encapsulated mRNA (mRNA F-LUC). The LNPS of the invention encapsulating mRNA F-LUC have been injected intravenously into a BALB/c mouse, at a dosage of 13 μg/mouse (0.52-0.65 mg/kg). In-vivo imaging were done by IVIS imaging. Ex-vivo tissue analysis was done for lung, heart, spleen, kidney, and liver using IVIS. Histology assessment was determined by H&E staining with pathologist report for toxicity. Histology assessment concluded normal morphology without any treatment related pathological changes.

The expression profile presented in FIG. 1 showed more than 100 times greater Luciferase signal in the lung as compared to heart, liver, spleen, and kidney, indicating superior lung specificity of the exemplary LNPs of the invention.

### Example 3

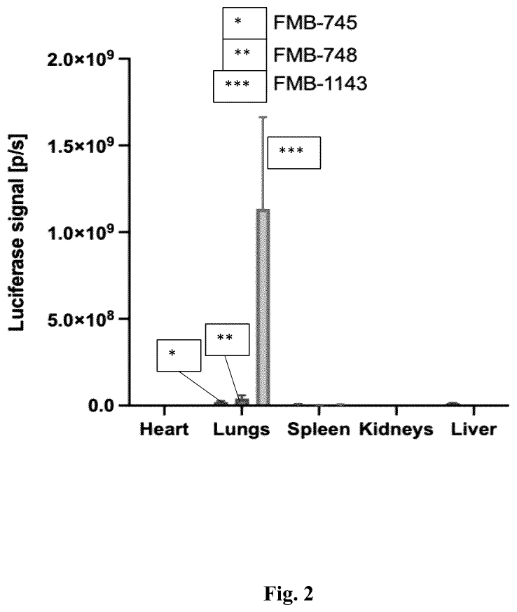

The inventors successfully prepared LNP formulations (FMB-428 and FMB-389) based on the exemplary ionizable lipids of the invention. FMB-428 and FMB-389 exhibited greater liver expression in-vivo (see FIG. 2), compared to similar LNP formulations based on commercial ionizable lipid (Dlin-MC3-DMA).

### Preparation of FMB-386, FMB-428, FMB-389, FMB-1050

10% of DOPE, 38.5% of cholesterol, 1.5% DMG-PEG2000 and 50% of ionizable MB-205, MB-222, MB-208, and Dlin-MC3-DMA respectively, were solubilized in ethanol (EtOH) at 55-60° C. mRNA, F-LUC was added to citrate buffer at pH of 5.0 (range 4.5-5.5). Mixing of lipids was done under microfluidic mixing or by EtOH injection of the lipids into the mRNA F-LUC containing citrate buffer under constant mixing conditions. pH of the mixture was then elevated using PBS dilution and residual EtOH was removed prior to injection using dialysis prepared by the inventors have been characterized by an average particle size ranging between about 60 and about 180 nm.

The inventors determined the expression distribution of the exemplary LNPs in vivo by assessing expression of the encapsulated mRNA (mRNA F-LUC). The LNPs of the invention encapsulating mRNA F-LUC have been injected intravenously into a BALB/c mouse, at a dosage of 13 μg/mouse (0.52-0.65 mg/kg). In-vivo imaging were done by IVIS imaging. Ex-vivo tissue analysis was done for lung, heart, spleen, kidney, and liver using IVIS. Histology assessment was determined by H&E staining with pathologist report for toxicity. Histology assessment concluded normal morphology without any treatment related pathological changes.

The results of this experiment are summarized in FIG. 2, showing enhanced liver expression in the liver tissue, as compared to a control LNP composition comprising a commercial “gold standard” ionizable lipid.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

### Example 4

Exemplary thioether-based compounds of the invention have been synthesized according to synthetic schemes presented hereinabove (Example 1).

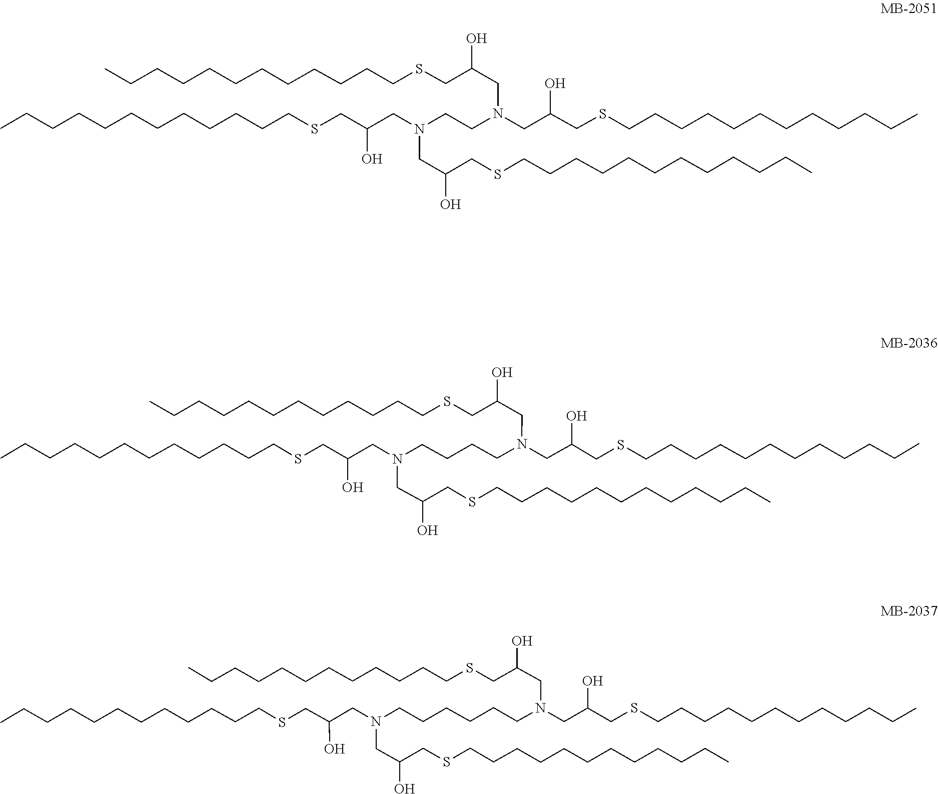

Based on in-silico prediction the inventor postulate that it is preferential utilizing above 10 mol % (e.g. between 15 and 60% mol, or between 20 and 55% mol) of the thioether-based compound of the invention to obtain the LNPs of the invention with an average particle size of between 50 and 180 nm with low PDI. The prediction demonstrated that under 10% by mol of the ionizable lipid of the invention within the formulation, results in LNPs with a high PDI values.

The inventor successfully utilized exemplary thiol compounds of the invention, MB-2036 and MB-2037, for the preparation of stable LNPs (FMB-1723, FMB-1724, FMB-1725, FMB-1726), with low PDI and an average particle size of between 80 and 110.

### Preparation of FMB-1723, FMB-1724, FMB-1725, FMB-1726

10% of DOPE, 38.5% of cholesterol, 1.5% DMG-PEG2000 and 50% of ionizable MB-2036 or MB-2037 were solubilized in ethanol (EtOH) at 55-60° C. mRNA, (F-LUC) was added to citrate buffer at pH of 5.0 (range 4.5-5.5). Mixing of lipids was done under microfluidic mixing or by EtOH injection of the lipids into the mRNA containing citrate buffer under constant mixing conditions to maintain N:P of 8 or 12. pH of the mixture was then elevated using PBS dilution. The LNPs have been characterized by an average particle size ranging between about 80 and about 92 nm.

1. A compound, a salt of said compound or both, wherein the compound is represented by Formula I: 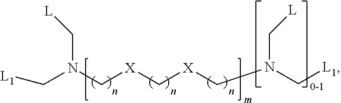
wherein: each L is independently R1, 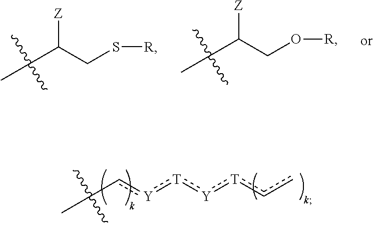
each L1 is independently R1, 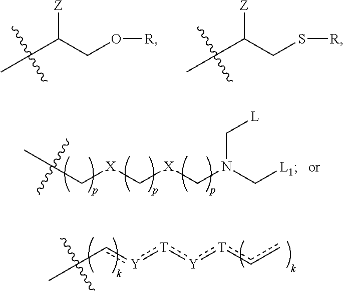 [symbol]
represents a single bond, a triple bond or a double bond;
each Z independently represents-OH or —SH;
each k is independently between 0 and 10;
each Y independently is absent or comprises CH₂, CHR′₂, NR′₂, NH, O, S, —CONH—, —CONR′—, —C(═NH)NR′—, —C(═S)NR′—, —NC(═O)—, —NC(═O)O—, —NC(═O)N—, —NC(═S)O—, —NC(═S)N—, —C(═O)—, —C(═O)O—, —OC(═O)O—, —OC(═O)N—, —OC(═S)O—, —OC(═S)N—, or phosphate, as allowed by valency;
each T independently represents an optionally substituted C5-C30 alkyl or an optionally substituted C5-C30 alkenyl;
each R′ is independently H or comprises an optionally substituted C₁-C₁₀ alkyl, an C₁-C₁₀ alkyl-aryl, an C₁-C₁₀ alkyl-cycloalkyl, optionally substituted C₃-C₁₀ cycloalkyl, optionally substituted C₃-C₁₀ heterocyclyl, optionally substituted heteroaryl, optionally substituted aryl or a combination thereof;
each X independently represents a heteroatom, CH₂, an optionally substituted C1-C10 alkyl, or X is absent;
each n and p is independently between 0 and 5, and at least one n is not 0;
m is between 1 and 3;
each R independently is H, or comprises an optionally substituted C5-C30 alkyl;
each R1 is an optionally substituted C1-C24 alkyl, and at least one L or L1 is or comprises

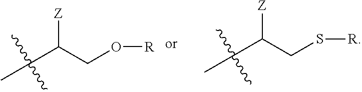

2. The compound of claim 1, wherein each L is independently 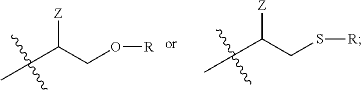
wherein any one of R and R1 further comprises at least one unsaturated bond; and wherein the heteroatom comprises O, N, NH, NR1, S, or a phosphate group.

3. (canceled)

4. (canceled)

5. The compound of claim 1, wherein each X independently is O, or is absent; and wherein any one of R and R1 represents a linear or a branched C1-C24 alkyl.

6. (canceled)

7. The compound of claim 1, wherein the compound is represented by Formula II: 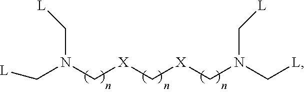
wherein each L is

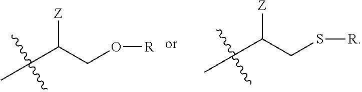

8. The compound of claim 1, wherein each R independently is an optionally substituted C5-C30 alkyl.

9. The compound of claim 1, wherein the compound is characterized by pKa value between 5 and 9.

10. A lipid nanoparticle comprising the compound of claim 1, and an active agent, wherein said active agent is a polynucleic acid.

11. The lipid nanoparticle of claim 10, wherein an average size of the lipid nanoparticle is in a range between 50 and 300 nm.

12. (canceled)

13. The lipid nanoparticle of claim 10, further comprising a lipid, wherein said lipid comprises a helper lipid, a structural lipid and a modified lipid.

14. The lipid nanoparticle of claim 13, wherein a weight ratio between (i) the total amount of the compound and of said lipid, and (ii) the polynucleic acid within said lipid nanoparticle is between 0.001:1 and 10:1.

15. The lipid nanoparticle of claim 13, wherein said structural lipid is a sterol.

16. The lipid nanoparticle of claim 10, wherein a ratio of the compound is between 10 and 80 mol %; a ratio of said structural lipid is between 5 and 50 mol %, a ratio of said helper lipid is between 10 and 50 mol %, and a ratio of said modified lipid is between 0.5 and 5 mol % relative to the total lipid content of the lipid nanoparticle.

17. The lipid nanoparticle of claim 16, wherein the lipid nanoparticle comprises between 20 and 60 mol % of said compound relative to the total lipid content of the lipid nanoparticle.

18. (canceled)

19. The lipid nanoparticle of claim 17, wherein the lipid nanoparticle consist essentially of: between about 20 and about 50 mol % of said compound, between about 20 and about 45 mol % of said structural lipid, between about 10 and about 50 mol % of said helper lipid, and between about 1 and about 3 mol % of said modified lipid relative to the total lipid content of the lipid nanoparticle; and wherein a N:P ratio within the lipid nanoparticle is between 5 and 12.

20. A pharmaceutical composition comprises a plurality of the lipid nanoparticles of claim 10 and a pharmaceutically acceptable carrier.

21. The pharmaceutical composition of claim 20, comprising an effective amount of the active agent; wherein said active agent is encapsulated within the lipid nanoparticle.

22. (canceled)

23. The pharmaceutical composition of claim 20, formulated for systemic administration to a subject, local administration to a subject, or both.

24. (canceled)

25. A method for delivering an active agent into a tissue of a subject, the method comprising administering to said subject an effective amount of the pharmaceutical composition of claim 20, thereby delivering the active agent to said tissue.

26. The method of claim 25, wherein said delivering comprises increasing a concentration of said active agent within said tissue, as compared to a control formulation comprising lipofectamine and said active agent and being devoid of the lipid nanoparticles; and wherein said increasing is by at least 10 times, as compared to the control formulation.

27. (canceled)
